(19) United States
(12) Patent Application Publication
(10) Pub. No.: US 2026/0281630 A1
(43) Pub. Date: Sep. 17, 2026

(54) HEARING AID FITTING

(71) Applicant: Tencent Technology (Shenzhen) Company Limited, Shenzhen (CN)

(72) Inventors: Cong FU, Shenzhen (CN); Guangqi ZHEN, Shenzhen (CN); Jun ZHAO, Shenzhen (CN); Chang LV, Shenzhen (CN); Shidong SHANG, Shenzhen (CN)

(73) Assignee: Tencent Technology (Shenzhen) Company Limited, Shenzhen (CN)

(21) Appl. No.: 19/664,988

(22) Filed: Apr. 30, 2026

Related U.S. Application Data
(63) Continuation of application No. PCT/CN2025/081547, filed on Mar. 10, 2025.

(30) Foreign Application Priority Data
Mar. 29, 2024 (CN) 202410392540.2

(51) Int. Cl. H04R 25/00

(52) U.S. Cl. CPC H04R 25/50; H04R 25/554; H04R 2225/41

(57) ABSTRACT

In a hearing aid fitting method, connection status information indicating a connection status between a second electronic device and a hearing aid is received by a first electronic device, the second electronic device being communicatively coupled to the hearing aid based on short-range wireless communication. In the method, a detection result indicating whether an environmental condition based on the connection status information satisfies a fitting condition is determined. In the method, a first fitting parameter for fitting the hearing aid is determined based on the connection status information, when the detection result indicates that the environmental condition satisfies the fitting condition. In the method, the first fitting parameter is transmitted to the second electronic device that is configured to engage in a fitting process with the hearing aid based on the first fitting parameter. Apparatus and non-transitory computer-readable storage medium counterparts are also contemplated.

### RELATED APPLICATIONS

The present application is a continuation of International Application No. PCT/CN2025/081547, filed on Mar. 10, 2025, which claims priority to Chinese Patent Application No. 202410392540.2, filed on Mar. 29, 2024. The entire disclosures of the prior applications are hereby incorporated by reference.

### FIELD OF THE TECHNOLOGY

This disclosure relates to the field of hearing aid fitting technologies, including hearing aid fitting methods and apparatuses, electronic devices, non-transitory computer-readable storage media, and computer program products.

### BACKGROUND OF THE DISCLOSURE

A hearing aid is an electronic device configured to help a user with hearing impairment improve hearing. The hearing aid is configured to amplify a sound signal, and the user with hearing impairment may hear a sound from outside more clearly, to improve a communication capability and quality of life.

In a related technology, in a process of fitting a hearing aid, a fitting user selects a proper hearing aid based on degree of hearing loss of the user with hearing impairment, and adjusts and optimizes the selected hearing aid. However, there is a problem of relatively low fitting accuracy in a process in which a fitting user fits a hearing aid online.

### SUMMARY

In view of this, embodiments of this disclosure provide hearing aid fitting methods and apparatuses, electronic devices, non-transitory computer-readable storage media, and computer program products, which can improve accuracy of fitting a hearing aid.

The embodiments of this disclosure provide a hearing aid fitting method. In the method, connection status information indicating a connection status between a second electronic device and a hearing aid is received by processing circuitry of a first electronic device, the second electronic device being communicatively coupled to the hearing aid based on wireless communication. In the method, a detection result indicating whether an environmental condition based on the connection status information satisfies a fitting condition is determined. In the method, a first fitting parameter for fitting the hearing aid is determined by the processing circuitry based on the connection status information, when the detection result indicates that the environmental condition satisfies the fitting condition. In the method, the first fitting parameter is transmitted to the second electronic device that is configured to engage in a fitting process with the hearing aid based on the first fitting parameter.

The embodiments of this disclosure provide a hearing aid fitting apparatus that includes processing circuitry configured to receive connection status information indicating a connection status between an electronic device and a hearing aid, the electronic device being communicatively coupled to the hearing aid based on wireless communication. The processing circuitry is configured to determine a detection result indicating whether an environmental condition based on the connection status information satisfies a fitting condition. The processing circuitry is configured to determine, based on the connection status information, a first fitting parameter for fitting the hearing aid when the detection result indicates that the environmental condition satisfies the fitting condition. The processing circuitry is configured to transmit the first fitting parameter to the electronic device that is configured to engage in a fitting process with the hearing aid based on the first fitting parameter.

The embodiments of this disclosure provide a non-transitory computer-readable storage medium storing instructions, which when executed by a processor, cause the processor to perform a hearing aid fitting method. In the method, connection status information indicating a connection status between a second electronic device and a hearing aid is received, the second electronic device being communicatively coupled to the hearing aid based on wireless communication. In the method, a detection result indicating whether an environmental condition based on the connection status information satisfies a fitting condition is determined. In the method, a first fitting parameter for fitting the hearing aid is determined based on the connection status information, when the detection result indicates that the environmental condition satisfies the fitting condition. In the method, the first fitting parameter is transmitted to the second electronic device that is configured to engage in a fitting process with the hearing aid based on the first fitting parameter.

The embodiments of this disclosure provide a hearing aid fitting method, the method being applied to a first electronic device, and the method including: receiving, by the first electronic device, connection status information transmitted by a second electronic device, the connection status information being configured to indicate a connection status between the second electronic device and a hearing aid, and the second electronic device being connected to the hearing aid by using short-range wireless communication; determining, based on the connection status information, a first fitting parameter for fitting the hearing aid; and transmitting the first fitting parameter to the second electronic device, the second electronic device being configured to fit, based on the first fitting parameter, the hearing aid.

The embodiments of this disclosure provide a hearing aid fitting apparatus. The apparatus includes: a receiving module, configured to receive, by a first electronic device, connection status information transmitted by a second electronic device, the connection status information being configured to indicate a connection status between the second electronic device and a hearing aid, and the second electronic device being connected to the hearing aid by using short-range wireless communication; a determining module, configured to determine, based on the connection status information, a first fitting parameter for fitting the hearing aid; and a transmitting module, configured to transmit the first fitting parameter to the second electronic device, to trigger the second electronic device to fit, based on the first fitting parameter, the hearing aid.

The embodiments of this disclosure provide a non-transitory computer-readable storage medium, having a computer program stored therein, and the computer program, when executed by processing circuitry (e.g., a processor), implements the foregoing hearing aid fitting method.

According to a fourth aspect of this disclosure, an electronic device is provided, including a memory (e.g., including a non-transitory computer-readable storage medium) and processing circuitry (e.g., a processor), the memory having a computer program stored therein, and the processor, when executing the computer program, implementing the foregoing hearing aid fitting method.

According to a fifth aspect of this disclosure, a computer program product is provided, including a computer program, the computer program being read and executed by processing circuitry (e.g., a processor) of an electronic device, to enable the electronic device to perform the foregoing hearing aid fitting method.

According to the hearing aid fitting method provided in one or more embodiments of this disclosure, a first electronic device may receive connection status information transmitted by a second electronic device, the connection status information being configured to indicate the connection status between the second electronic device and the hearing aid, and the second electronic device being connected to the hearing aid by using the short-range wireless communication; determine, based on the connection status information, the first fitting parameter for fitting the hearing aid; and transmit the first fitting parameter to the second electronic device, to trigger the second electronic device to fit, based on the first fitting parameter, the hearing aid.

In a fitting process of the hearing aid, the first fitting parameter for fitting the hearing aid may be determined with reference to information that indicates the connection status between the second electronic device and the hearing aid. In some aspects, a fitting parameter may be dynamically adjusted according to the connection status between the second electronic device and the hearing aid. In some aspects, the fitting parameter based on the connection status between the second electronic device and the hearing aid may be usable for fitting the hearing aid. This may reduce problems of inaccurate fitting of the hearing aid caused by an unstable connection status between the second electronic device and the hearing aid, and may be compatible with processes of fitting hearing aids in different connection statuses, and to improve accuracy of fitting the hearing aids.

### BRIEF DESCRIPTION OF THE DRAWINGS

Accompanying drawings are used to facilitate understanding of examples in this disclosure, and constitute a part of the disclosure.. The accompanying drawings are not intended to limit this disclosure.

FIG. 1 is an architectural diagram of a system to which a hearing aid fitting method is applied according to an embodiment of this disclosure.

FIG. 2 is a schematic flowchart of a hearing aid fitting method according to an embodiment of this disclosure.

FIG. 3 is a schematic diagram in which a first electronic device displays a Bluetooth list according to an embodiment of this disclosure.

FIG. 4 is a schematic diagram in which a first electronic device transmits a fitting request to a second electronic device according to an embodiment of this disclosure.

FIG. 5 is another schematic diagram in which a first electronic device transmits a fitting request to a second electronic device according to an embodiment of this disclosure.

FIG. 6 is a schematic diagram in which a first electronic device displays a Bluetooth connection success according to an embodiment of this disclosure.

FIG. 7 is a schematic diagram of a left ear audiometry result curve according to an embodiment of this disclosure.

FIG. 8 is a schematic diagram of a right ear audiometry result curve according to an embodiment of this disclosure.

FIG. 9 is a schematic flowchart of a Bluetooth connection method according to an embodiment of this disclosure.

FIG. 10 is another schematic flowchart of a hearing aid fitting method according to an embodiment of this disclosure.

FIG. 11 is a schematic diagram of data transmission in a hearing aid fitting process according to an embodiment of this disclosure.

FIG. 12 is a schematic structural diagram of a hearing aid fitting apparatus according to an embodiment of this disclosure.

FIG. 13 is a schematic structural diagram of an electronic device according to an embodiment of this disclosure.

### DETAILED DESCRIPTION

To illustrate objectives, technical solutions, and advantages of this disclosure, the following describes this disclosure with reference to accompanying drawings. Described examples are not to be considered as a limitation to this disclosure. All other aspects obtained by a person of ordinary skill in the art fall within the scope of this disclosure.

Descriptions of terms in this disclosure are provided as examples and are not intended to limit the scope of the disclosure.

In the following descriptions, "some embodiments" describes a subset of all possible embodiments. However, the "some embodiments" may be the same subset or different subsets of all the possible embodiments, and may be combined with each other without conflict.

Unless otherwise defined, all technical and scientific terms used herein have the same meanings as commonly understood by those skilled in the art belonging to this disclosure. Terms used herein are merely intended to describe the embodiments of this disclosure, but are not intended to limit this disclosure.

FIG. 1 is an architectural diagram of a system to which a hearing aid fitting method is applied according to an embodiment of this disclosure. The system includes a first electronic device 110, a second electronic device 120, and a hearing aid 130. The first electronic device 110 corresponds to a fitting end, and the second electronic device 120 corresponds to a client.

The first electronic device 110 includes a plurality of device forms such as a desktop computer, a laptop computer, a personal digital assistant (PDA), a mobile phone, an in-vehicle terminal, a home theater terminal, a dedicated terminal, a smart voice interaction device, a smart home appliance, or an aircraft. In addition, the first electronic device may be a single device, or may be a set including a plurality of devices. The first electronic device 110 may communicate with the second electronic device 120 in a wired or wireless manner, to exchange data.

The second electronic device 120 includes a plurality of device forms such as a desktop computer, a laptop computer, a PDA, a mobile phone, an in-vehicle terminal, a home theater terminal, a dedicated terminal, a smart voice interaction device, a smart home appliance, or an aircraft. In addition, the second electronic device may be a single device, or may be a set including a plurality of devices.

The hearing aid 130 may be classified into an air conduction hearing aid and a bone conduction hearing aid according to a conduction manner. The hearing aid is classified into a cassette type, a glasses type, a hairpin type, a behind-the-ear type, an in-the-ear type, a canal type, and a deep canal type hearing aid according to a use manner. A type and a brand of the hearing aid 130 are not limited herein.

The hearing aid fitting method provided in this disclosure may be implemented in the first electronic device 110. In some examples, the hearing aid fitting method provided in this disclosure is applied to the first electronic device. The second electronic device 120 is connected to the hearing aid 130 by using short-range wireless communication. The first electronic device 110 controls the second electronic device 120 to fit the hearing aid 130.

In some aspects, the hearing aid fitting method provided in this disclosure is implemented in the first electronic device 110. The first electronic device 110 receives connection status information transmitted by the second electronic device 120. The first electronic device 110 determines, based on the connection status information, a first fitting parameter for fitting the hearing aid 130. The first electronic device 110 transmits the first fitting parameter to the second electronic device 120, to trigger the second electronic device 120 to fit, based on the first fitting parameter, the hearing aid 130.

The hearing aid fitting method provided in this disclosure may be applied to various hearing aid fitting scenarios, or may be compatible with different hearing aid types. In some embodiments, the hearing aid fitting method provided in this disclosure may implement remote fitting of a hearing aid. For example, a distance between the first electronic device and the hearing aid is greater than a first distance threshold.

For ease of understanding, the following describes an application scenario of the hearing aid fitting method provided in this disclosure. First, for some users who are in relatively remote geographical locations and cannot get to an offline physical store for fitting due to a distance factor, the hearing aid fitting method provided in this disclosure may be used. Second, for some users who intend to seek comfortable fitting experience and intend to complete hearing aid fitting without leaving their homes, the hearing aid fitting method provided in this disclosure may be used. Third, for users who pay more attention to personal privacy and do not want to cause leakage of personal privacy due to offline fitting and need to fit at home or in a relatively private environment, the hearing aid fitting method provided in this disclosure may be used. Fourth, for some users who have mobility impairments or need to keep a social distance for personal reasons, the hearing aid fitting method provided in this disclosure may be used. In some aspects, the scenario may alternatively be another scenario, and may be determined according to an actual use requirement.

The hearing aid fitting method provided in this disclosure may be applied to one or more of the foregoing scenarios. In some aspects, a fitting process is controlled by the first electronic device. The first electronic device receives the connection status information transmitted by the second electronic device, determines, based on the connection status information, the first fitting parameter for fitting the hearing aid, and transmits the first fitting parameter to the second electronic device, to trigger the second electronic device to fit, based on the first fitting parameter, the hearing aid. The foregoing examples do not limit the protection scope of this case.

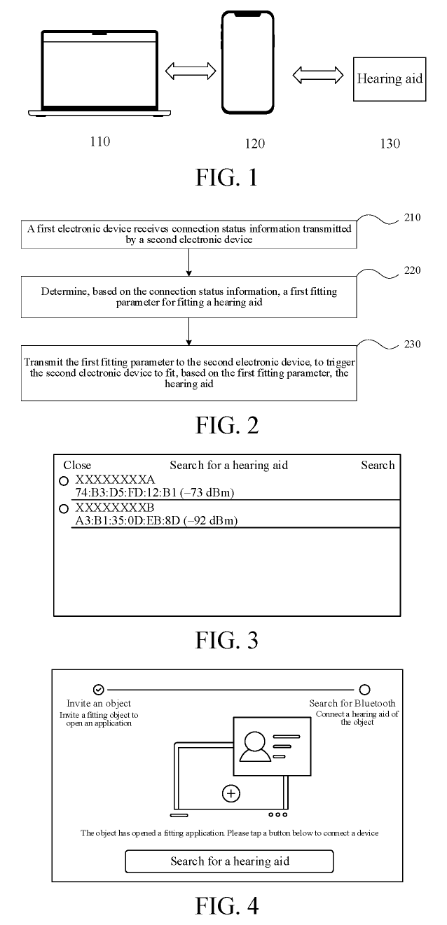

According to an embodiment of this disclosure, a hearing aid fitting method is provided. FIG. 2 is a schematic flowchart of a hearing aid fitting method according to an embodiment of this disclosure. The method may be applied to a hearing aid fitting apparatus. The hearing aid fitting apparatus may be integrated into an electronic device. The electronic device may be a terminal, a server, or a combination thereof. The hearing aid fitting method may include the following operations:

Operation 210: A first electronic device receives connection status information transmitted by a second electronic device. For example, connection status information indicating a connection status between a second electronic device and a hearing aid is received by processing circuitry of a first electronic device, the second electronic device being communicatively coupled to the hearing aid via short-range wireless communication.

The hearing aid fitting method provided in this disclosure is operated by a fitting user (e.g., a fitter) on the first electronic device. In some aspects, the hearing aid fitting method provided in this disclosure may be implemented on the first electronic device.

In a related technology, a user with hearing impairment is a user who needs to fit a hearing aid, and is merely used for distinguishing from a fitting user herein. The user with hearing impairment needs to manually connect to the second electronic device by using a Bluetooth hearing aid, and a connection status between the second electronic device and the hearing aid is of great importance to a hearing aid fitting process. If the connection status indicates that a connection between the second electronic device and the hearing aid fails, the hearing aid may not be fitted. In a fitting process of the hearing aid, if the connection status indicates that the second electronic device is disconnected from the hearing aid, a fitting interruption of the hearing aid is caused, and the second electronic device needs to be reconnected to the hearing aid.

For a case that a distance between the first electronic device and the second electronic device is greater than a first distance threshold, for example, for a case that the distance between the fitting user and the user with hearing impairment is greater than the first distance threshold, the first electronic device may not receive information of the fitting interruption in time, and the fitting user may not learn the information of the fitting interruption either.

In the related technology, the connection status has an impact on receiving audiometry audio by the second electronic device. The audiometry audio may be referred to as an audiometry corpus. When a fixed fitting parameter (including a parameter such as a playback speed of the audiometry corpus) is used to perform audiometry on a user using a hearing aid, an inaccurate audiometry result (for example, a fitting result) may be caused, and an inaccurate setting of a configuration parameter of the hearing aid may further be caused. Inaccurate setting of the configuration parameter of the hearing aid may cause poor hearing performance of the user with hearing impairment during use of the hearing aid, and may further damage hearing of the user.

However, according to the hearing aid fitting method provided in this disclosure, the first electronic device may acquire the connection status information in real time, and the fitting user may learn the connection status between the second electronic device and the hearing aid more quickly. In addition, the first fitting parameter for fitting the hearing aid may be dynamically adjusted with reference to the connection status information in a hearing aid fitting process. In some examples, fitting the hearing aid by using the fitting parameter suited to the connection status between the second electronic device and the hearing aid. The hearing aid fitting method provided in this disclosure may reduce problems in a related technology, and may improve accuracy of fitting the hearing aid.

In this disclosure, the second electronic device may be a smartphone, a tablet device, or a computer of the user using the hearing aid (e.g., the user with hearing impairment). This is not limited herein. The hearing aid is a hearing aid that needs to be fitted, and the hearing aid may be a non-debugged hearing aid purchased by a user offline or online. The hearing aid may alternatively be a hearing aid on which basic parameters and functions have been debugged, and parameter optimization needs to be performed at a time interval.

The connection status information is configured to indicate a connection status between the second electronic device and the hearing aid. The second electronic device is connected to the hearing aid by using short-range wireless communication. The connection status may include whether the second electronic device is connected to the hearing aid. In a case that the second electronic device is connected to the hearing aid, the connection status information may include one or more parameters that indicate the connection status. The connection status information is described below with reference to different short-range wireless communication technologies.

In some embodiments, the short-range wireless communication technology may be Bluetooth. Correspondingly, in a case that the second electronic device is connected to the hearing aid, the connection status information may include a signal strength, a connection interval, a connection timeout, electric quantity information, and the like. The signal strength is indicated by using a received signal strength indicator (RSSI). The connection interval refers to a time interval of performing data transmission between the second electronic device and the hearing aid. The connection timeout refers to maximum duration of a connection between the second electronic device and the hearing aid in a case that data is not transmitted. The electric quantity information may be electric quantity information of the second electronic device or electric quantity information of the hearing aid.

The signal strength is described below. A unit of the RSSI (the signal strength) is dBm, may be a negative value, and reflects attenuation degree of a signal. In an ideal state without attenuation, the signal strength may be 0 dBm. However, in an actual application process, even if the distance between the second electronic device and the hearing aid is less than a second distance threshold, and the second distance threshold is less than the first distance threshold, the signal strength may be -50 dBm, and loss may exist. In some embodiments, the connection status may be set as stable if the signal strength is greater than the first signal strength threshold. For example, the connection status may be set as stable if the signal strength is greater than -67 dBm.

For Bluetooth, factors affecting a Bluetooth signal strength between the hearing aid and the second electronic device include: a distance between the hearing aid and the second electronic device, a physical obstacle (such as a wall or a building) between the hearing aid and the second electronic device, a multi-path effect, another wireless device within a preset range, an electromagnetic interference source, another electronic device within a preset range, and the like. The connection interval and the connection timeout may be adjusted according to an actual use requirement. The multi-path effect means that a signal is reflected to form a plurality of paths, which may cause phase cancellation, to reduce the signal strength.

In some embodiments, the short-range wireless communication technology may be wireless network communication (Wi-Fi). The connection status information may include a signal strength, a signal to noise ratio (SNR), a connection rate, and the like. The SNR is a ratio of the signal strength to a background noise level. A higher SNR indicates a more stable connection status between the second electronic device and the hearing aid. The connection rate refers to a data transmission rate between the second electronic device and the hearing aid.

For Wi-Fi, factors affecting a signal strength and an SNR of the Wi-Fi between the hearing aid and the second electronic device include: a distance between the hearing aid and the second electronic device, a physical obstacle between the hearing aid and the second electronic device, another wireless device within a preset range, another electronic device within a preset range, and the like. Factors affecting the SNR of the Wi-Fi between the hearing aid and the second electronic device further include a selected channel. Factors affecting the connection rate between the hearing aid and the second electronic device include an SNR, a channel width, compatibility of the second electronic device and the hearing aid, and the like.

In some embodiments, the short-range wireless communication technology may be ZigBee, and the connection status information may include a signal strength and a network delay. The network delay refers to a delay time for data transmission between the second electronic device and the hearing aid.

For the ZigBee, factors affecting a signal strength of the ZigBee between the hearing aid and the second electronic device include: a distance between the hearing aid and the second electronic device, a physical obstacle between the hearing aid and the second electronic device, a multi-path effect, another wireless device within a preset range, an electromagnetic interference source, another electronic device within a preset range, and the like. Factors affecting the network delay of the ZigBee between the hearing aid and the second electronic device include: a network configuration, a channel characteristic, a transmission mechanism, and the like.

A manner in which the second electronic device transmits the connection status information is described below. In some embodiments, the second electronic device may encapsulate the connection status information, and the first electronic device communicates with the second electronic device through a wireless data transmission channel, and then the first electronic device may receive the connection status information transmitted by the second electronic device.

In some embodiments, the wireless data transmission channel may be an instant message channel. The instant message channel has real-time performance and high efficiency. An instant message application supports online state display, which is beneficial to determining whether the other party is online and acquiring feedback in time. In a fitting process of the hearing aid, the first electronic device may instantly transmit a fitting message to the second electronic device by using the instant message channel, and the first electronic device may instantly acquire feedback from the second electronic device.

Information transmitted by the instant message may include binary information, plain text information, audio and video information, and the like. The instant message channel is configured to encapsulate, transmit, and decapsulate messages of the second electronic device and the first electronic device and may facilitate low-delay communication and reliable message transfer. A communication protocol used by the instant message may be an extensible messaging and presence protocol (XMPP), a session initialization protocol (SIP), or a real-time messaging protocol (RTMP). In some embodiments, the first electronic device and an object second electronic device may both be online conference second electronic devices.

In some embodiments, before receiving, by the first electronic device, the connection status information transmitted by the second electronic device, the method further includes:

transmitting, by the first electronic device, a fitting request to the second electronic device, the fitting request being configured to trigger the second electronic device to initiate a device search process compliant with a wireless communication protocol, such as short-range wireless communication protocol (e.g., Bluetooth search). The fitting request may be used as a fitting instruction. In some aspects, the first electronic device may transmit the fitting instruction to the second electronic device, and the second electronic device may initiate the Bluetooth search in response to the fitting instruction.

In some embodiments, the fitting request may be transmitted by using the instant message. After receiving the fitting request transmitted by the first electronic device, the second electronic device decapsulates the fitting request and parses a message, to initiate the Bluetooth search.

After initiating the device search process, the second electronic device may acquire one or more pieces of search information of content within a preset range. Then, the second electronic device may return the one or more pieces of search information to the first electronic device. The first electronic device may receive the one or more pieces of search information transmitted by the second electronic device, where the one or more pieces of search information are search information found in a case that the object second electronic device initiates the device search process.

In some embodiments, a plurality of pieces of search information may be displayed on a display screen of the first electronic device, the plurality of pieces of search information including a target piece of search information corresponding to a hearing aid; and a connection request is generated based on the target piece of search information corresponding to the hearing aid in response to a selection operation on the target piece of search information corresponding to the hearing aid.

When the plurality of pieces of search information include the target piece of search information corresponding to the hearing aid, the connection request is generated based on the target piece of search information corresponding to the hearing aid, and the connection request is transmitted to the second electronic device, to trigger the second electronic device to establish a short-range wireless connection compliant with the short-range wireless communication protocol (e.g., a Bluetooth connection) with the hearing aid based on the target piece of search information corresponding to the hearing aid in the connection request. The connection request may be a Bluetooth connection instruction in some aspects. In some aspects, the first electronic device may transmit the Bluetooth connection instruction to the second electronic device, and the second electronic device may establish a Bluetooth connection with the hearing aid based on the Bluetooth connection instruction.

In some embodiments, after receiving the fitting request, the second electronic device may display, on a display screen of the second electronic device, notification information for indicating that the second electronic device is establishing a Bluetooth connection with the hearing aid. The notification information may further include information of the hearing aid. In this way, based on the notification information, the information of the hearing aid and that the hearing aid is establishing the Bluetooth connection with the second electronic device may be learned.

In some embodiments, the notification information may further include recommendation information for improving stability of the connection status. The recommendation information may be "Please get close to the hearing aid", and the "Please get close to the hearing aid" is configured to recommend a user to shorten a distance between the second electronic device and the hearing aid. The recommendation information may further be "Far away from a physical obstacle and an electromagnetic interference source". The recommendation information may alternatively be "Choose a relatively quiet environment for fitting". In an example, the recommendation information may be set according to an actual use requirement.

Correspondingly, the user may consciously move, based on the recommendation information, a to-be-fitted hearing aid close to the second electronic device, and far away from the physical obstacle and the electromagnetic interference source, and choose a relatively quiet environment for fitting, to improve a success rate of controlling, by the first electronic device, the second electronic device to be connected to the hearing aid. For the Wi-Fi and the ZigBee, similar replacements may be made to the foregoing descriptions. For example, Bluetooth may be replaced with the Wi-Fi or the ZigBee, whereby the first electronic device controls the second electronic device to be connected to the hearing aid.

In some embodiments, in a process of displaying the notification message on the display screen of the second electronic device, the user may further be notified with reference to any one of vibration or ringtone, to further improve a success rate of controlling, by the first electronic device, the second electronic device to be connected to the hearing aid.

The following describes a manner of starting the hearing aid. In some embodiments, the fitting user and the user with hearing impairment may perform an audio call or a video call, whereby the user with hearing impairment may start the hearing aid under guidance of the fitting user, and then the user with hearing impairment may be guided to activate the Bluetooth of the hearing aid.

In some embodiments, for a case that the fitting user and the user with hearing impairment may perform the audio call or the video call, the fitting user may guide the user with hearing impairment to activate a Bluetooth search function of the second electronic device, and then the second electronic device may search for a device that is broadcasting around.

In some embodiments, the second electronic device searches for broadcasting of a Bluetooth-activated device around by using a generic access profile (GAP). The GAP is a sub-protocol defined in a Bluetooth low energy (BLE) technology, defines how devices discover each other, establish a connection, and implement binding, describes how devices become a broadcaster and an observer and implement data transmission without connection, and further defines different types of addresses to protect data privacy and address parsability.

In some embodiments, the second electronic device may transmit information of the hearing aid to the first electronic device. The information of the hearing aid may be one or more of a unique identifier character string of the hearing aid, a name of the hearing aid, and a model of the hearing aid. The first electronic device receives the information of the hearing aid transmitted to the second electronic device, and transmits a start request to the hearing aid based on the information of the hearing aid. The hearing aid is further configured to activate the Bluetooth based on the start request.

In some aspects, after the second electronic device initiates the Bluetooth search, the second electronic device acquires the Bluetooth information of the Bluetooth-activated device within the preset range. The Bluetooth information may include a Bluetooth name, a Bluetooth address, a Bluetooth signal strength, and the like. There may be one or more pieces of Bluetooth information acquired by the second electronic device.

The Bluetooth addresses displayed by the second electronic device and the first electronic device are represented by 12 hexadecimal characters, and the characters are separated by ":". For example, the Bluetooth address may be "00:0C:3E:3A:4B:69". The second electronic device encapsulates the found Bluetooth information, and transmits the Bluetooth information to the first electronic device in a form of an instant message. The first electronic device receiving the Bluetooth information transmitted by the second electronic device may be displaying the Bluetooth information in the first electronic device in a form of a pop-up window.

In some embodiments, after receiving the encapsulated Bluetooth information, the first electronic device may encapsulate and parse the encapsulated Bluetooth information, and display, on an interface of the first electronic device, the Bluetooth information obtained through parsing. In this manner, the fitting user may intuitively learn the Bluetooth information or the parsed Bluetooth information.

In some embodiments, a correspondence between the Bluetooth information and the hearing aid is preset. After the Bluetooth information is acquired, at least one of a name of the hearing aid, an image of the hearing aid, or a model of the hearing aid may be determined based on the Bluetooth information, and then the at least one of the name of the hearing aid, the image of the hearing aid, or the model of the hearing aid may be displayed on the display interface of the first electronic device, whereby richness of content displayed on the display interface may be improved, and the fitting user may more intuitively learn a hearing aid that needs to be fitted.

In some embodiments, when the plurality of pieces of Bluetooth information include the Bluetooth information of the hearing aid, the first electronic device transmits a connection request to the second electronic device, and the second electronic device may establish a Bluetooth connection with the hearing aid based on the connection request.

In some embodiments, a Bluetooth list generated based on the plurality of pieces of Bluetooth information may be displayed on the display screen of the first electronic device. The Bluetooth list includes the Bluetooth information for the hearing aid, and the display screen may further display a connection control.

In response to a selection operation for the Bluetooth information of the hearing aid, the Bluetooth information of the hearing aid may be controlled to be in a selected state. Based on the Bluetooth information in the selected state, a connection request may be generated based on the Bluetooth information of the hearing aid in response to a trigger operation for the connection control, and then the connection request may be transmitted to the second electronic device. The second electronic device may establish a Bluetooth connection with the hearing aid based on the Bluetooth information of the hearing aid in the connection request.

In some embodiments, after parsing the received plurality of pieces of Bluetooth information, the first electronic device displays the parsed Bluetooth information in a form of a to-be-connected Bluetooth list on a fitting software interactive interface of the first electronic device.

FIG. 3 is a schematic diagram in which a first electronic device displays a Bluetooth list according to an embodiment of this disclosure. A Bluetooth list is displayed in FIG. 3, and the list displays a Bluetooth search result, (for example, a plurality of pieces of Bluetooth information). FIG. 3 shows a device named "XXXXXXXXA", with a corresponding Bluetooth address of "74:B3:D5:FD:12:B1", and a signal strength of a Bluetooth connection of "-73 dBm", and a device named "XXXXXXXXB", with a corresponding Bluetooth address of "A3:B1:35:0D:EB:8D", and a signal strength of a Bluetooth connection of "-92 dBm".

The fitting user may determine, according to the Bluetooth name or the Bluetooth address, whether the Bluetooth list includes the Bluetooth information of the hearing aid. If the Bluetooth list includes the Bluetooth information of the hearing aid, a connection request may be generated based on the Bluetooth information of the hearing aid in response to a selection operation of the fitting user for the Bluetooth information of the hearing aid. The first electronic device may transmit the connection request to the second electronic device. The second electronic device may establish a Bluetooth connection with the hearing aid based on the Bluetooth information of the hearing aid in the connection request.

If the Bluetooth list does not include the Bluetooth information of the hearing aid, a search control may be displayed on a display interface of the first electronic device. A fitting request may be generated in response to a trigger operation for the search control, then the first electronic device may transmit the fitting request to the second electronic device, and the second electronic device may initiate Bluetooth search again.

In some embodiments, for the connection request, the first electronic device may combine the Bluetooth information of the hearing aid and signaling word information for the Bluetooth connection, encapsulate the combined information into a binary or plain text form to obtain encapsulated information, and generate the connection request based on the encapsulated information. The first electronic device transmits the generated connection request to the second electronic device in a form of an instant message. A signaling word is a control signal for controlling a communication process.

After receiving the connection request transmitted by the first electronic device, the second electronic device may decapsulate and parse the connection request, to obtain the Bluetooth information of the hearing aid and the signaling word information for the Bluetooth connection. The second electronic device may establish a Bluetooth connection with the hearing aid according to a GAP protocol based on the Bluetooth information of the hearing aid and the signaling word information for the Bluetooth connection.

In some embodiments, before transmitting, by the first electronic device, the fitting request to the second electronic device, the hearing aid fitting method provided in this disclosure further includes:

receiving, by the first electronic device, the fitting request of the hearing aid transmitted by the second electronic device. The first electronic device may determine the hearing aid based on the fitting request of the hearing aid. The fitting request of the hearing aid includes information of the hearing aid. After the hearing aid is determined, the first electronic device may transmit a Bluetooth activation instruction to the hearing aid. The hearing aid may start based on the Bluetooth activation instruction and broadcast a Bluetooth signal to the outside. In this manner, the first electronic device may control the hearing aid to activate Bluetooth of the hearing aid.

In some embodiments, the fitting request of the hearing aid may include a model, a type, a device number of the hearing aid, a version of fitting software of the second electronic device, and the like. After receiving the fitting request of the hearing aid, the first electronic device may determine the hearing aid for which Bluetooth needs to be activated.

In some embodiments, the first electronic device transmits the fitting request to the second electronic device. After receiving the fitting request, the second electronic device may initiate Bluetooth search, and transmit a Bluetooth activation instruction to the hearing aid.

FIG. 4 is a schematic diagram in which a first electronic device transmits a fitting request to a second electronic device according to an embodiment of this disclosure. In the figure, an "Invite an object" control displays that the second electronic device has opened fitting application software. The first electronic device may transmit the fitting request to the second electronic device in response to a trigger operation for a "Search for a hearing aid" control, and the second electronic device may transmit a Bluetooth activation instruction to the hearing aid, to activate the Bluetooth. The object second electronic device activates a Bluetooth adapter, to search for Bluetooth-activated devices within a preset range. An object is a user with hearing impairment. In this manner, the Bluetooth of the hearing aid does not need to be manually activated by the user with hearing impairment, to improve fitting efficiency of the hearing aid, and improve use experience for the user with hearing impairment.

In some embodiments, the second electronic device may transmit a power-on instruction to the hearing aid based on the fitting application software. The hearing aid may be automatically powered on in response to the power-on instruction. In this manner, a to-be-fitted hearing aid does not need to be manually turned on by the user with hearing impairment, and the hearing aid may be controlled to be started by the second electronic device, to improve use experience for the user with hearing impairment.

In some embodiments, the second electronic device transmits a power-on instruction to the hearing aid in a wireless communication protocol, and the hearing aid starts after receiving the power-on instruction. In some embodiments, in a case that the second electronic device receives the fitting request, the power-on instruction and the Bluetooth activation instruction may be transmitted to the hearing aid in a wireless communication protocol, whereby the hearing aid may receive the power-on instruction and the Bluetooth activation instruction; the hearing aid is started in response to the power-on instruction; and the Bluetooth is activated in response to the Bluetooth activation instruction (for example, a Bluetooth signal broadcasting mode is entered).

After activating the Bluetooth, the hearing aid may transmit Bluetooth activation information to the second electronic device. The second electronic device may transmit the Bluetooth activation information to the first electronic device. After receiving the Bluetooth activation information, the first electronic device may display "The object hearing aid has been started" on the display interface in a form of a pop-up window, or may display "The object hearing aid has been started, and Bluetooth search may be performed" on the display interface. In an example, the foregoing text may alternatively be played.

If the hearing aid fails to be powered on, or the hearing aid does not activate the Bluetooth, activation failure information may be generated. In some embodiments, the activation failure information may be transmitted by the hearing aid to the second electronic device, or may be automatically generated after the second electronic device does not receive feedback information of the hearing aid within preset duration. The second electronic device may transmit the activation failure information to the first electronic device, the first electronic device may display the activation failure information, and then the first electronic device may retransmit the fitting request. In this manner, a fitting user may learn, in real time, a progress of fitting a hearing aid, to improve accuracy of fitting the hearing aid.

In some embodiments, after activating the Bluetooth, the hearing aid starts to broadcast Bluetooth information. The Bluetooth information may include a Bluetooth name, a Bluetooth address, and a Bluetooth signal strength. The Bluetooth information may be transmitted to the second electronic device by using the GAP.

In some embodiments, after activating the Bluetooth, the hearing aid may further transmit device information of the hearing aid to the second electronic device by using the GAP. The device information of the hearing aid may be information such as an electric quantity, a speech, and a volume of the hearing aid.

The second electronic device transmits the device information of the hearing aid to the first electronic device by using an instant message channel, and the first electronic device can transmit, for a current performance state of the hearing aid, corresponding notification information to a client. For example, in a case that the electric quantity is lower than an electric quantity threshold, the first electronic device may generate electric quantity notification information. The electric quantity notification information may be that: the electric quantity is insufficient, please charge the hearing aid.

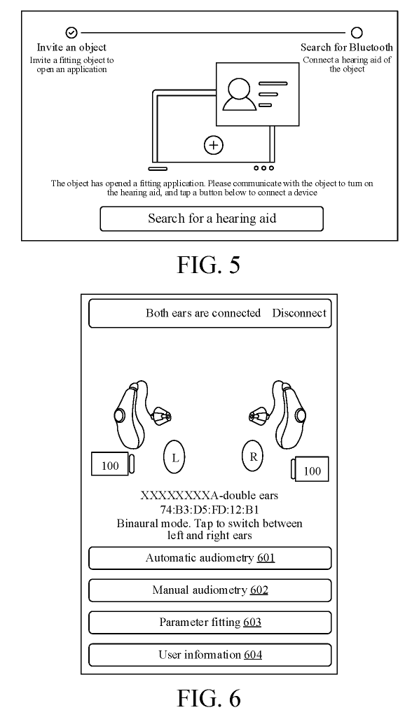

FIG. 5 is a schematic diagram in which a first electronic device transmits a fitting request to a second electronic device according to an embodiment of this disclosure. In this case, the fitting user may communicate with the user with hearing impairment by using real-time video software or call software. Notification information may be displayed in FIG. 5. The notification information is "Please communicate with an object to turn on the hearing aid". The fitting user may guide the user with hearing impairment to start the hearing aid, to facilitate controlling a Bluetooth connection between the second electronic device and the hearing aid by the first electronic device.

In some embodiments, after the second electronic device establishes a Bluetooth connection with the hearing aid, an interactive interface of fitting application software of the first electronic device is updated. FIG. 6 is a schematic diagram in which a first electronic device displays a Bluetooth connection success according to an embodiment of this disclosure. FIG. 6 shows connection statuses of a left-ear hearing aid and a right-ear hearing aid. The left-ear hearing aid and the right-ear hearing aid shown in FIG. 6 are both in selectable states. In response to a selection operation on at least one of the left-ear hearing aid, the right-ear hearing aid, or both, the at least one of the left-ear hearing aid, the right-ear hearing aid, or both is controlled to be in a selected state, and the hearing aid in the selected state may be fitted. For example, separate fitting may be implemented for the left-ear hearing aid and the right-ear hearing aid, or joint fitting may be implemented for the left-ear hearing aid and the right-ear hearing aid. In an example, setting may be performed according to an actual use requirement, to improve selectability and accuracy of fitting the hearing aid.

In FIG. 6, remaining electric quantities of the left-ear hearing aid and the right-ear hearing aid may be further displayed, to facilitate real-time detection of the electric quantity. If the user with hearing impairment wears only a single hearing aid, the hearing aid worn by the user may be controlled to be in a selected state, to implement fitting for the hearing aid that is in the selected state.

In FIG. 6, a device name "XXXXXXXXA" and a Bluetooth address "74:B3:D5:FD:12:B1" of a connected to-be-fitted hearing aid may be further displayed. The left-ear hearing aid and the right-ear hearing aid may be switched by tapping an icon corresponding to the left-ear hearing aid and an icon corresponding to the right-ear hearing aid, thereby implementing fitting for a single ear or double ears.

In some embodiments, FIG. 6 may further display a plurality of fitting controls, which are respectively automatic audiometry 601, manual audiometry 602, parameter fitting 603, and user information 604. An audiometry mode may be automatically switched in response to a trigger operation for the automatic audiometry 601; audiometry may be manually performed in response to a trigger operation for the manual audiometry 602; and a configuration parameter for a hearing aid may be set in response to a trigger operation for the parameter fitting 603.

The automatic audiometry 601 is configured to perform audiometry by using a computer technology and audiometry software. Automatic audiometry software provides a plurality of test programs, such as pure tone audiometry, speech audiometry, and audiometry in noise. A user with hearing impairment or a fitting user may select an appropriate test program according to an actual situation. The first electronic device automatically records a hearing threshold of the user with hearing impairment and draws an audiogram curve according to a test result.

The manual audiometry 602 is configured to enable the fitting user to gradually adjust, according to a preset test procedure and requirement, intensity and frequency of audio played by the hearing aid, acquire a response of the user with hearing impairment, record the hearing threshold, and draw the audiogram curve.

The user information 604 may be configured to check and record the audiogram curve and the hearing threshold, and may further record a model of the hearing aid, a brand of the hearing aid, age, gender, and degree of hearing impairment of the user with hearing impairment, to facilitate learning, by the fitting user, information of the user with hearing impairment corresponding to the hearing aid, and improving accuracy of fitting the hearing aid.

Operation 220: Determine, based on the connection status information, a first fitting parameter for fitting a hearing aid. In some examples, a detection result indicating whether an environmental condition based on the connection status information satisfies a fitting condition is determined. In some examples, a first fitting parameter for fitting the hearing aid is determined by the processing circuitry based on the connection status information, when the detection result indicates that the environmental condition satisfies the fitting condition.

In some embodiments, environment detection is performed based on the connection status information, to obtain a detection result. The first fitting parameter for fitting the hearing aid is determined based on the connection status information in a case that the detection result indicates that an environmental condition satisfies a fitting condition.

In some embodiments, whether an environment in which the hearing aid is located meets a fitting requirement may be determined first. For a case that a distance between the first electronic device and the second electronic device is greater than a first distance threshold, (e.g., the hearing aid is fitted remotely), the fitting user may not be able to predict and adjust the environment in which the user with hearing impairment is located (e.g., the environment in which the hearing aid is located), resulting in relatively low accuracy of fitting the hearing aid.

To improve the accuracy of fitting of the hearing aid, a first signal strength threshold of the hearing aid may be acquired in a case that the connection status information includes a first signal strength, environment detection is performed based on the first signal strength and the first signal strength threshold, and, in a case that the first signal strength is greater than the first signal strength threshold, a detection result is set to indicate that the environmental condition satisfies the fitting condition. For example, the detection result is set to indicate that the environmental condition satisfies the fitting condition when the first signal strength is greater than a first signal strength threshold associated with the hearing aid.

The detection result is that the environmental condition satisfies a not-fitting condition in a case that the first signal strength is less than or equal to the first signal strength threshold. In some aspects, in a case that the detection result indicates that the environmental condition does not satisfy the fitting condition, guidance information may be generated based on the connection status information, the guidance information being configured to indicate an adjustment to a relative position between the second electronic device and the hearing aid. The guidance information is transmitted to the second electronic device, to trigger the second electronic device to display the guidance information. In some embodiments, the guidance information is recommendation information. For the guidance information, refer to descriptions for the recommendation information.

In some embodiments, the first electronic device may set the first signal strength threshold in advance, where the first signal strength threshold is a signal strength reference value of Bluetooth, and the first signal strength is a signal strength of a Bluetooth connection between the hearing aid and the second electronic device. For example, the first electronic device sets the first signal strength threshold to -60 dBm. After receiving the first signal strength of the Bluetooth connection between the hearing aid and the second electronic device, the first electronic device may automatically determine whether the first signal strength is greater than the first signal strength threshold.

In some embodiments, when the first signal strength is greater than the first signal strength threshold, stability of the Bluetooth connection in this case is relatively good, hence a current fitting environment result may be determined as satisfying a fitting requirement, and fitting may be performed.

When the first signal strength is not greater than the first signal strength threshold, the stability of the Bluetooth connection between the current to-be-fitted hearing aid and an object client is poor. In this case, performing fitting is prone to Bluetooth disconnection, and an interruption of a fitting process is caused. In this case, the fitting user may assist the user with hearing impairment in analyzing a reason why the environmental condition does not satisfy the fitting condition.

In some embodiments, the first electronic device may sequentially transmit different guidance information to the second electronic device. For example, the first electronic device may first transmit guidance information 1 to the second electronic device. The guidance information 1 is configured to guide the user with hearing impairment to move the hearing aid close to the second electronic device, and the user with hearing impairment moves the hearing aid close to the second electronic device.

The first electronic device may acquire the connection status information in real time. After first duration, if the connection status information indicates that the environmental condition still does not satisfy a fitting condition, the first electronic device may transmit guidance information 2 to the second electronic device. The guidance information 2 may be at least one of "far away from a physical obstacle", "far away from an electromagnetic interference device", or "far away from noise". In some aspects, other guidance information may alternatively be transmitted according to an actual use requirement, until the environmental condition satisfies the fitting condition. In this manner, the first electronic device may determine, according to the connection status information received in real time, whether the environmental condition satisfies the fitting condition, whereby a case that a Bluetooth connection status is unstable may be discovered in time, and reliability of fitting the hearing aid remotely may be improved. For the fitting condition, refer to descriptions of parameters affecting short-range wireless communication.

In the hearing aid fitting method provided in this embodiment of this disclosure, environment detection may be performed based on the connection status information, and a user may be guided, by using the guidance information, to perform fitting in a quiet environment, to get away from an electronic device having electromagnetic interference, and to get away from an obstacle such as a wall, to improve the first signal strength, and improve reliability of fitting the hearing aid remotely.

In some embodiments, the guidance information may be a pose adjustment instruction. In a case that the detection result indicates that the environmental condition does not satisfy the fitting condition, the pose adjustment instruction for a relative pose between the second electronic device and the to-be-fitted hearing aid may be generated, and the pose adjustment instruction is transmitted to the second electronic device. The second electronic device displays content corresponding to the pose adjustment instruction, or the second electronic device may play a pose adjustment notification voice corresponding to the pose adjustment instruction.

In some embodiments, the relative pose between the second electronic device and the hearing aid includes a distance between the second electronic device and the hearing aid and a placement position of the hearing aid relative to the second electronic device. The first electronic device generates the pose adjustment instruction for the relative pose between the second electronic device and the to-be-fitted hearing aid. The pose adjustment instruction is configured to instruct the user to move the to-be-fitted hearing aid close to the second electronic device, or adjust the placement position of the hearing aid relative to the second electronic device.

After generating the pose adjustment instruction, the first electronic device encapsulates the pose adjustment instruction into binary information or plain text information, and transmits the pose adjustment instruction to the second electronic device through an instant message channel. After receiving the pose adjustment instruction, the second electronic device parses the pose adjustment instruction, and the second electronic device displays content corresponding to the pose adjustment instruction, or the second electronic device may play a pose adjustment notification voice corresponding to the pose adjustment instruction.

In some embodiments, the connection status information may further include a packet loss rate between the second electronic device and the hearing aid, a data transmission rate between the second electronic device and the hearing aid, electric quantity, and the like. The signal strength and the packet loss rate are configured to indicate stability of a Bluetooth connection. The data transmission rate affects the data transmission rate between the second electronic device and the hearing aid, and further affects a data transmission rate between the second electronic device and the first electronic device. When the data transmission rate becomes slow, the second electronic device may be unable to receive, in time, a fitting parameter and audio transmitted by the first electronic device.

When the first signal strength is relatively weak (the first signal strength is less than or equal to the first signal strength threshold), strength of audio that is transmitted by the first electronic device to the second electronic device, then transmitted by the second electronic device to the hearing aid, and output by the hearing aid weakens accordingly, or a part of the audio is unclear, whereby the user may not understand the audio, and a playback speed of the audio changes.

When the first signal strength is relatively weak (the first signal strength is less than or equal to the first signal strength threshold), audio data actually received by the second electronic device and audio data output from the hearing aid are greatly different from audio data transmitted by the first electronic device, to finally result in reduction of accuracy of an audiometry result (e.g., a fitting result), hearing performance verification (e.g., verification of performance of a configured hearing aid), and parameter configuration of the hearing aid. Consequently, accuracy of fitting the hearing aid is reduced.

For a case that the detection result indicates that the environmental condition satisfies the fitting condition, the first fitting parameter for fitting the hearing aid may be determined based on the connection status information. The first fitting parameter may be a playback speed of the audio, a frequency response of the audio, a reverberation effect of the audio, or the like, and may be set according to an actual use requirement.

In some embodiments, a correspondence between the playback speed of the audio and the signal strength is preset. A playback speed corresponding to the first signal strength may be determined based on the first signal strength in the connection status information, and then the playback speed corresponding to the first signal strength and audiometry audio may be transmitted to the second electronic device. The second electronic device may transmit the playback speed corresponding to the first signal strength and the audiometry audio to the hearing aid.

In some embodiments, in a case that the detection result indicates that the environment satisfies the fitting condition, the reverberation effect corresponding to the connection status information may be determined according to the correspondence between the connection status information and the reverberation effect, whereby the first electronic device may transmit audiometry audio having the reverberation effect corresponding to the connection status information and the reverberation effect corresponding to the connection status information to the second electronic device, and the second electronic device may transmit the audiometry audio having the reverberation effect corresponding to the connection status information to the hearing aid.

In a case that the detection result indicates that the environmental condition satisfies the fitting condition, the connection status information may include an audio encoding format of Bluetooth, and a frequency response corresponding to the audio encoding format included in the connection status information may be determined based on a correspondence between the audio encoding format and the frequency response, whereby the first electronic device may transmit audiometry audio having the frequency response corresponding to the audio encoding format and the frequency response corresponding to the audio encoding format to the second electronic device, and the second electronic device may transmit the audiometry audio having the frequency response corresponding to the audio encoding format to the hearing aid.

The hearing aid may play the received audiometry audio. The second electronic device may display feedback questions for the audiometry audio, may acquire answers to the feedback questions in response to an answer input operation of the user with hearing impairment based on the feedback question, and may generate a fitting result based on the answers to the feedback questions. The feedback questions may have an association relationship with the frequency response corresponding to the audio encoding format, the reverberation effect corresponding to the connection status information, and the playback speed corresponding to the first signal strength.

In some embodiments, the connection status information includes a first signal strength, and the operation that "Determine, based on the connection status information, a first fitting parameter for fitting the hearing aid" may be implemented in the following manners:

acquiring a second signal strength threshold and a second fitting parameter of the hearing aid, where the second signal strength threshold may be the same as or different from the first signal strength threshold, and may be set according to an actual use requirement; and

determining a ratio of the first signal strength to the second signal strength threshold, and determining, based on the ratio and the second fitting parameter, the first fitting parameter for fitting the hearing aid. In some embodiments, the second fitting parameter includes a first playback speed of the audiometry audio, a second playback speed of the audiometry audio may be determined based on the first playback speed and the ratio, and the first fitting parameter for fitting the hearing aid is determined based on the second playback speed.

In some embodiments, the second signal strength threshold may be a reference Bluetooth signal strength. The second signal strength threshold may be a signal strength value that may improve accuracy of fitting the hearing aid in an actual fitting process, and may be set according to an experience of fitting the hearing aid remotely by the fitting user, or may be set according to a uniform signal strength standard in the field of hearing aid fitting.

For example, -50 dBm may be used as the second signal strength threshold, which may satisfy a signal strength required for fitting the hearing aid and may also facilitate calculation of a fitting parameter. After setting the second signal strength threshold, the first electronic device may receive the first signal strength transmitted by the second electronic device, and calculate a ratio of the first signal strength to the second signal strength threshold.

In some embodiments, the second fitting parameter includes a first playback speed (for example, a reference playback speed) of the audiometry audio. The second playback speed of the audiometry audio may be determined based on the first playback speed and the ratio. The first fitting parameter for fitting the hearing aid may be determined according to the second playback speed of the audiometry audio.

In some embodiments, the audiometry audio refers to an audio material used in audiometry, and is configured to test a hearing level and an understanding capability of the user with hearing impairment. The audiometry audio usually includes the following types: a pure tone signal, a speech signal, an ambient sound, a music signal, and a noise signal.

The first playback speed of the audiometry audio is a playback speed that is most proper for the user with hearing impairment in a case that the first signal strength is greater than the first signal strength threshold, and a Bluetooth data transmission rate reaches a preset rate.

In a case that the first signal strength is less than or equal to the first signal strength threshold, the audiometry audio needs to be transmitted and processed between the second electronic device and the hearing aid for a longer time. Therefore, the playback speed of the audiometry audio transmitted to the hearing aid is reduced.

In some embodiments, the second playback speed of the audiometry audio is calculated according to the first signal strength and the first playback speed. The second playback speed may better match a hearing adaptability of the user with hearing impairment at the first signal strength, and improve accuracy of the fitting parameter for fitting the hearing aid, whereby the user with hearing impairment achieves a better hearing experience.

In some embodiments, the first fitting parameter may be determined based on a relationship between the first signal strength and the second signal strength. In a case that the first signal strength is greater than the second signal strength, the first playback speed may not be adjusted. For example, if the second signal strength threshold is set to -60 dBm, the first signal strength is set to -40 dBm, and the first signal strength is greater than the second signal strength threshold, a current Bluetooth connection status is relatively stable, and good transmission of the audiometry audio may be facilitated. The first playback speed may not be adjusted; for example, the first playback speed may be determined as the first fitting parameter.

In some embodiments, the second playback speed of the audiometry audio may be calculated according to the ratio of the first signal strength to the second signal strength threshold and the first playback speed of the audiometry audio. If the second signal strength threshold is -50 dBm, and the first signal strength is -75 dBm, the first electronic device automatically calculates that the ratio of the first signal strength to the second signal strength threshold is two thirds.

In this case, the first signal strength is lower than the second signal strength threshold, and the strength of the audiometry audio transmitted by the first electronic device to the second electronic device and the hearing aid may be weakened. During transmission, partial audio may be distorted, and the audiometry audio transmitted to the second electronic device may have a noise. If the audiometry audio is played at a normal speed, the user with hearing impairment may not accurately feed back content of the audiometry audio, to result in an inaccurate hearing understanding capability of the user with hearing impairment in an audiometry result.

To improve the accuracy of fitting the hearing aid, the first playback speed may be adjusted based on the ratio of the first signal strength to the second signal strength threshold. The first playback speed is reduced by one third based on the ratio of the first signal strength to the second signal strength threshold, to obtain the second playback speed, and then the first fitting parameter for fitting the hearing aid may be determined based on the second playback speed.

For example, if the first playback speed is 1× speed, 1 may be multiplied by two thirds. In some aspects, this reduces the first playback speed by one third. Therefore, the second playback speed may be two thirds of the speed. In this manner, the first playback speed may be dynamically adjusted based on the relationship between the first signal strength and the second signal strength threshold, to obtain the second playback speed, and then the second playback speed may be combined with one or more of the reverberation effect and the audio frequency response, to obtain the first fitting parameter for fitting the hearing aid, thereby helping to ensure that the user with hearing impairment may better understand the audiometry audio, and improving accuracy of an audiometry result (a fitting result).

In this disclosure, the first fitting parameter of the hearing aid that better conforms to the connection status information may be obtained, and the hearing aid may play the audiometry audio based on the first fitting parameter, to perform audiometry on the user with hearing impairment, and obtain a more accurate fitting result.

In some embodiments, the connection status information includes a third signal strength of short-range wireless communication at a first moment and a fourth signal strength of the short-range wireless communication at a second moment. The first moment is earlier than the second moment. In some aspects, signal strengths at a plurality of different moments may be included, and a quantity may be set according to an actual use requirement, and details are not described herein again.

A difference between the third signal strength and the fourth signal strength is acquired. The first fitting parameter for fitting the hearing aid is determined based on the fourth signal strength in a case that the difference is greater than a difference threshold, and the first fitting parameter for fitting the hearing aid is determined based on the third signal strength in a case that the difference is less than or equal to the difference threshold. For a specific determining manner, refer to the foregoing description. In this manner, a more accurate fitting parameter may be acquired, to improve accuracy of fitting the hearing aid.

In some embodiments, the connection status information includes a plurality of fifth signal strengths. Different fifth signal strengths are at different moments. An average value of the plurality of fifth signal strengths may be acquired, and then the first fitting parameter for fitting the hearing aid may be determined based on the average value of the fifth signal strengths. For a specific determining manner, refer to the foregoing description. In this manner, a more accurate fitting parameter may be acquired, to improve accuracy of fitting the hearing aid.

Operation 230: Transmit the first fitting parameter to the second electronic device, to trigger the second electronic device to fit, based on the first fitting parameter, the hearing aid. For example, the first fitting parameter is transmitted to the second electronic device that is configured to engage in a fitting process with the hearing aid based on the first fitting parameter.

In some embodiments, the first electronic device may transmit the first fitting parameter to the second electronic device, to trigger the second electronic device to fit, based on the first fitting parameter, the hearing aid. In some embodiments, the first electronic device may transmit both the audiometry audio and the first fitting parameter to the second electronic device. The second electronic device may transmit the audiometry audio and the first fitting parameter to the hearing aid, and then the hearing aid may play the audiometry audio based on the audiometry audio and the first fitting parameter.

In some embodiments, the first electronic device may transmit only the first fitting parameter to the second electronic device. The second electronic device may select, based on the first fitting parameter, the audiometry audio from audio pre-stored in the second electronic device. The second electronic device may transmit the audiometry audio and the first fitting parameter to the hearing aid, and then the hearing aid may play the audiometry audio based on the audiometry audio and the first fitting parameter.

In some embodiments, the first electronic device may receive a fitting result returned by the second electronic device. The first electronic device may generate a configuration parameter of the hearing aid based on the fitting result, and transmit the configuration parameter of the hearing aid to the second electronic device, to trigger the second electronic device to configure the hearing aid based on the configuration parameter of the hearing aid.

In some embodiments, audiometry may be performed on a hearing aid wearer, and a parameter of the hearing aid is configured according to a result of the audiometry (for example, a fitting result). The audiometry may include subjective audiometry and objective audiometry. In the subjective audiometry, minimum intensity of a sound that can be heard by human ears may be measured by using an artificially generated pure tone or language speech of different intensities and different frequencies. A subject expresses a response to a sound signal by using a main complaint, a gesture, or a control indicator. Content of this method includes: whisper check, stopwatch check, tuning fork check, pure tone audiometry, and speech audiometry. In the objective audiometry, a physiological response, an acoustic impedance phenomenon, and a bioelectric phenomenon that are caused after a subject receives a sound signal are used as indexes for determining a hearing function state. Such methods are not affected by subjective factors, and check results are relatively accurate.

In some embodiments, the first electronic device transmits the first fitting parameter, including the second playback speed of the audiometry audio, to the second electronic device. After receiving the second playback speed, the second electronic device initiates audiometry according to the second playback speed.

The fitting result may be acquired by using a neural response or a cochlear response of ears of the user with hearing impairment to the audiometry audio, or whether a current test object is able to hear a sound of normal volume, whether there is noise, whether a speed is suitable, and whether hearing is comfortable may be determined by using real-time feedback of the user, such as oral feedback and facial expression.

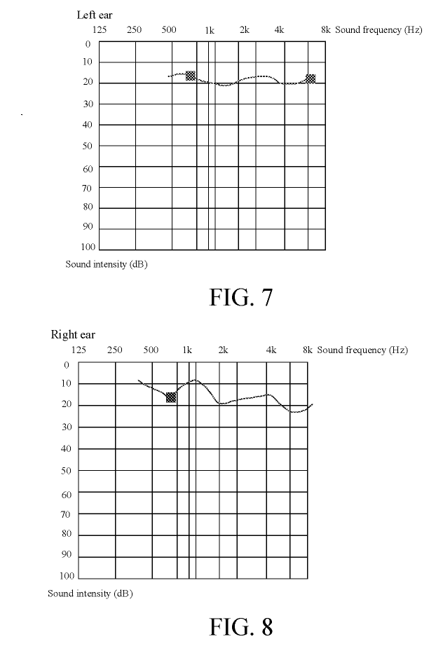

In some embodiments, the second electronic device transmits the audiometry result to the first electronic device. An audiometry result is usually displayed in a form of an audiogram curve. FIG. 7 is a left ear audiometry result curve according to an embodiment of this disclosure, and FIG. 8 is a right ear audiometry result curve according to an embodiment of this disclosure.

For FIG. 7 and FIG. 8, the horizontal coordinates represent a frequency of a sound, which is measured in Hertz (Hz). Vertical coordinates indicate represent an intensity of a sound, and may alternatively represent a hearing level, which is measured in decibel (dB) and represents degree of hearing loss. An audiogram curve may indicate a hearing threshold of a test object in sounds of different frequencies, and the fitting user may adjust a configuration parameter of the hearing aid according to the audiogram curve. In addition, an audiometry result may further include audiograms obtained by performing an air conduction test and a bone conduction test on the test object, including some symbols for representing test results. For example, the bone conduction test is performed on a left ear, no masking is represented by using ">", and masking is represented by using "]".

In some embodiments, the first electronic device may generate a configuration parameter of a hearing aid based on a fitting result transmitted by the second electronic device. The configuration parameter may include: a gain, a frequency response, a saturated sound pressure level, a frequency range, noise suppression, and the like. The gain represents a capability of a hearing aid to amplify a sound, and hearing may be compensated by using hearing aids with different gains according to different degrees of hearing loss. To meet a hearing requirement, the hearing aid provides different frequency responses, and different frequencies reflect different tones in hearing. To make the frequency response of the hearing aid meet a characteristic of a hearing loss of an object with hearing impairment, some different tones are set on a tone adjustment button.

The frequency range is a frequency range of a sound that may be heard by the object with hearing impairment. If the object with hearing impairment has a poor hearing experience at a sound frequency, a configuration parameter of the hearing aid is adjusted, and the hearing aid may perform gain compensation on a sound signal at this frequency, to restore the hearing of the object with hearing impairment at this sound frequency. The saturated sound pressure level is maximum output power of a hearing aid. Setting a proper saturated sound pressure level may provide better hearing compensation performance for the object with hearing impairment, and damage to hearing of the user due to excessively high intensity of an output sound may be reduced. The noise suppression may be to suppress noise generated by a hearing aid, or may alternatively suppress noise interference in an ambient environment. Adjustment of a noise suppression parameter may enable the object with hearing impairment to hear a clearer target sound.

In some embodiments, verification audio is determined based on the connection status information, and an encoding parameter for the verification audio is determined, the verification audio being configured to verify performance of the hearing aid configured by using the configuration parameter; and a verification request is generated based on the verification audio and the encoding parameter. In some embodiments, the verification audio is compressed based on the encoding parameter to obtain a compression result, and the verification request including the compression result is generated. The encoding parameter may include a sampling rate, an encoding bit rate, a bit depth, and the like.

The sampling rate is a quantity of times of collecting an audio signal of the verification audio per second, the encoding bit rate is a data volume of the audio data of the verification audio transmitted or processed in a unit time, and the bit depth is a number of binary bits included in each sampling point. The sampling rate determines the frequency range and reconstruction precision of an audio signal, the encoding bit rate determines compression degree and transmission efficiency of audio data, and the bit depth determines a dynamic range and tone quality of an audio signal.

In some embodiments, the verification request is transmitted to the second electronic device, to trigger the second electronic device to verify performance of the configured hearing aid based on the verification audio in the verification request. The foregoing process may be repeatedly performed. The foregoing processing process for the verification audio may alternatively be performed for the audiometry audio, and similar replacement may be performed on corresponding content. Details are not described herein again.

In some embodiments, a verification result returned by the second electronic device may be received. A manner of acquiring the verification result may be the same as a manner of acquiring the fitting result. For further details, refer to corresponding descriptions. When the verification result indicates that hearing aid performance of the hearing aid is good, a fitting process is terminated. In some aspects, this completes the fitting for the hearing aid.

In a case that the verification result indicates that the performance of the hearing aid is poor, the fitting user may reset a configuration parameter of the hearing aid, to obtain an updated configuration parameter, and then transmit the updated configuration parameter to the second electronic device, thereby configuring the hearing aid by using the updated configuration parameter, then determining the verification audio based on the connection status information, generating the verification request based on the verification audio and the encoding parameter, and transmitting the verification request to the second electronic device, to trigger the second electronic device to verify the performance of the configured hearing aid based on the verification audio in the verification request, until the verification result indicates that the hearing aid performance of the hearing aid is good.

In some embodiments, after configuring the hearing aid according to the configuration parameter of the hearing aid, the second electronic device may verify the hearing aid performance of the configured hearing aid. The verification audio for the hearing aid performance may include a voice material, a music material, an ambient sound material, and an ambient noise material. In an actual verification process, proper verification audio may be selected for verification according to an actual hearing requirement and a characteristic of a hearing aid.

A playback speed of the verification audio is updated according to a first signal strength. After determining the verification audio, the first electronic device may generate the verification request based on the verification audio and the encoding parameter, to transmit the verification request to the second electronic device. The verification request includes compressed verification audio (for example, a compression result).

The second electronic device initiates hearing aid performance verification on the hearing aid according to the compressed verification audio. In some embodiments, the signal strength changes. To improve accuracy of fitting a hearing aid, a second signal strength transmitted by the second electronic device may be periodically received. The second signal strength is transmitted by the second electronic device in a fitting process of the hearing aid based on the first fitting parameter.

A difference between the second signal strength and the first signal strength is determined, a third fitting parameter is generated when the difference is greater than a preset difference threshold, and the third fitting parameter is transmitted to the second electronic device, to trigger the second electronic device to replace the first fitting parameter with the third fitting parameter. A method for generating the first fitting parameter may be replaced with a method for generating the third fitting parameter, and details are not described herein again.

When the first signal strength changes, the first electronic device determines the verification audio and determines an encoding bit rate for the verification audio. During transmission of the verification audio, the verification audio may be encoded, and the verification audio is compressed into an audio bitstream, to reduce a data volume of the verification audio and reducing costs of data transmission and storage. The encoding bit rate of the verification audio is a proportion of a useful information part in the audio bitstream after sampling, quantizing, and encoding of an audio signal are completed. When the verification audio is transmitted to the second electronic device, the audio bitstream is further decoded, to restore original verification audio.

In this embodiment of this disclosure, the encoding bit rate for encoding the verification audio may be determined according to the first signal strength. The encoding bit rate is an encoding bit rate of the verification audio transmitted by the first electronic device to the second electronic device. The encoding bit rate may alternatively be an encoding bit rate of the verification audio transmitted by the second electronic device to the hearing aid.

In some embodiments, when the first signal strength is relatively low, the encoding bit rate for encoding the verification audio may be reduced, whereby the hearing aid may receive complete verification audio; and when a Bluetooth signal strength is relatively high, the encoding bit rate for encoding the verification audio may be increased. Thus, the hearing aid may obtain verification audio of higher quality, to obtain a more accurate verification result.

In some embodiments, the verification of the hearing aid performance is to verify an output gain, a frequency range, and a noise reduction capability of the hearing aid, and to analyze a speech recognition capability of a user with hearing impairment after wearing the hearing aid. Analyzing the output gain of the hearing aid is to verify whether a gain setting of the hearing aid matches a hearing loss level of the user with hearing impairment, and the frequency range is analyzed to verify whether the hearing aid may satisfy a hearing loss existing at some frequencies. The noise reduction capability may reflect a capability of a hearing aid to suppress self-noise and ambient noise, and verifying the speech recognition capability of the user with hearing impairment may analyze whether the user with hearing impairment may hear and understand a meaning expressed by a sound material. After performing hearing aid performance verification on the hearing aid, the second electronic device obtains a hearing aid performance verification result (for example, a verification result). As the audiometry result (the fitting result) described in the foregoing operations, the hearing aid performance verification result may alternatively be represented by using an audiogram curve. The second electronic device transmits the obtained hearing aid performance verification result back to the first electronic device.

In some embodiments, the first electronic device receives the verification result of the hearing aid performance. The fitting user analyzes the verification result. If the hearing aid performance verification result indicates a good hearing performance, for example, the output gain of the hearing aid may well compensate for a hearing loss of the user with hearing impairment, and may well reduce noise interference, with the help of the hearing aid, the user with hearing impairment may well understand information conveyed by another person by speaking. In this case, the fitting process may be terminated, and the Bluetooth connection between the second electronic device and the hearing aid is disconnected.

In some embodiments, the periodically receiving the second signal strength transmitted by the second electronic device is receiving the second signal strength that is of a short-range wireless communication connection between the second electronic device and the hearing aid and that is returned by the second electronic device at preset duration.

When the difference is greater than the preset difference threshold, a third fitting parameter is generated, and the third fitting parameter is transmitted to the second electronic device, to trigger the second electronic device to replace the first fitting parameter with the third fitting parameter.

In some embodiments, the second electronic device may transmit the second signal strength to the first electronic device at preset duration. The preset duration may be several seconds or several minutes. This is not limited herein. The first electronic device may determine a connection status between the second electronic device and the hearing aid according to the second signal strength, to reduce an interruption of a fitting process caused by sudden disconnection midway.

In some embodiments, when the first electronic device receives the second signal strength, the first electronic device subtracts the second signal strength from the first signal strength to obtain a difference, and a magnitude of a change of a signal strength may be detected by using the difference. If the second signal strength is lower than the first signal strength, the second signal strength is subtracted from the first signal strength to obtain a difference. If the difference is greater than a preset difference threshold, a current connection status is unstable, and the fitting parameter needs to be updated; and if the difference is less than or equal to the preset difference threshold, the connection status is stable, and the fitting parameter does not need to be updated.

In some embodiments, a playback speed of audio may be reduced. For example, the first signal strength is -50 dBm, the second signal strength is -60 dBm, the preset difference threshold is 5 dBm, the difference between the first signal strength and the second signal strength is 10 dBm, and the difference is greater than the preset threshold. A third playback speed needs to be calculated according to the method for calculating the second playback speed in the foregoing operation, and the third fitting parameter is determined based on the third playback speed.

In some embodiments, if the second signal strength is higher than the first signal strength, the first signal strength is subtracted from the second signal strength to obtain the difference. If the difference is greater than the preset difference threshold, the connection status is stable. If the audio is played at the playback speed included in the first fitting parameter, uncomfortable hearing may be generated. If the difference is not greater than the preset difference threshold, the fitting parameter does not need to be updated.

For example, if the first signal strength is -50 dBm, and the second signal strength is -40 dBm, the difference is 10 dBm, and is greater than the preset threshold 5 dBm. In this case, the fitting parameter needs to be updated, for example, a playback speed is properly increased. In this manner, the signal strength may be acquired in real time, and the fitting parameter is updated based on the signal strength, to improve accuracy of fitting the hearing aid.

After generating the third fitting parameter, the first electronic device may transmit the third fitting parameter to the second electronic device. The second electronic device receives the third fitting parameter, and continues to fit the hearing aid according to the third fitting parameter. The fitting parameter may be automatically updated according to the signal strength, to improve accuracy of an audiometry result, and improve accuracy of fitting the hearing aid.

According to this embodiment of this disclosure, the first electronic device controls the second electronic device to be connected to the hearing aid, then updates, according to signal strengths of the second electronic device and the hearing aid, a fitting parameter for fitting the hearing aid, and fits the hearing aid based on the fitting parameter. In this manner, a more accurate fitting result and verification result may be acquired, to acquire a more accurate configuration of the hearing aid, and improve accuracy of a hearing aid fitting system.

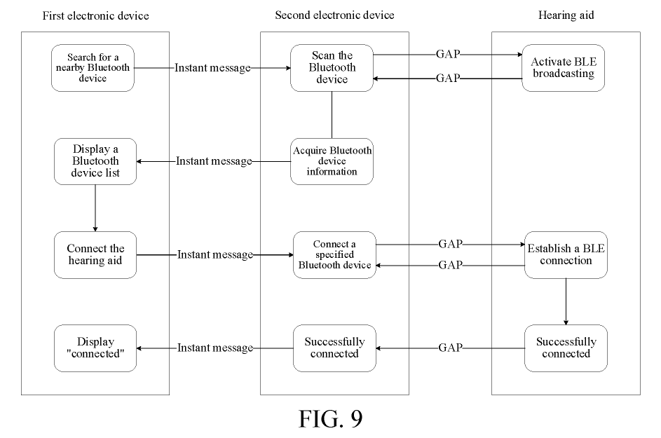

FIG. 9 is a schematic flowchart of a Bluetooth connection method according to an embodiment of this disclosure. In this embodiment, a scenario in which a person with hearing impairment in a remote region fits a hearing aid is used as an example to describe the hearing aid fitting method in further detail with reference to an execution subject of operations. A method for remotely controlling, by the first electronic device, the second electronic device to establish a Bluetooth connection with the hearing aid is as follows:

Before the Bluetooth connection is established, the first electronic device may acquire, by using the second electronic device, user information such as a model, a brand, and a basic parameter of a to-be-fitted hearing aid and a Bluetooth address corresponding to the to-be-fitted hearing aid. A touch key of the user information is displayed on a fitting application interface of the first electronic device. When the to-be-fitted hearing aid in an available Bluetooth device list is searched subsequently, a fitting user may query information of the to-be-fitted hearing aid by tapping the touch key of the user information.

The first electronic device transmits a power-on instruction of the hearing aid to the second electronic device, and the second electronic device controls, according to the power-on instruction, the hearing aid to start, or may notify, by using a real-time audio communication technology, a user to turn on the hearing aid. After the hearing aid is powered on, Bluetooth broadcasting is activated to broadcast Bluetooth information of the hearing aid to the outside. The first electronic device encapsulates an instruction for searching for a nearby Bluetooth device into an instant message, and transmits the instant message to the second electronic device. The second electronic device decapsulates and parses the received instant message, and activates a Bluetooth adapter, to scan surrounding Bluetooth devices that are broadcasting. The second electronic device searches, by using the GAP, for a nearby Bluetooth device that is broadcasting, and acquires Bluetooth information of surrounding Bluetooth devices.

In some embodiments, the second electronic device encapsulates the Bluetooth device information and transmits the Bluetooth device information to the first electronic device in a form of an instant message. The first electronic device receives the Bluetooth information, decapsulates and parses the Bluetooth information, and displays the Bluetooth device list on a software fitting interactive interface. If the Bluetooth device list includes the Bluetooth information of the to-be-fitted hearing aid, the first electronic device initiates a Bluetooth connection instruction (for example, a connection request) for connecting to the to-be-fitted hearing aid, and transmits the Bluetooth connection instruction to the second electronic device by using an instant message channel.

After receiving the Bluetooth connection instruction, the second electronic device parses the instruction information, to obtain a Bluetooth address of a to-be-fitted hearing aid, and transmits the Bluetooth connection instruction to the to-be-fitted hearing aid. The second electronic device is connected to a specified to-be-fitted hearing aid device according to the GAP, and the to-be-fitted hearing aid responds to the Bluetooth connection instruction of the second electronic device, establishes a BLE connection, and transmits Bluetooth response information to the second electronic device by using the GAP.

After responding to the Bluetooth connection status information of the second electronic device, the to-be-fitted hearing aid transmits, by using the GAP, information indicating a connection success to the second electronic device. The second electronic device receives the information indicating that the second electronic device establishes a Bluetooth connection with the to-be-fitted hearing aid, and a fitting application interface of the second electronic device may display a message notification indicating that the second electronic device is connected to the to-be-fitted hearing aid, encapsulate the information, and return the information to the first electronic device in a form of an instant message. The first electronic device receives the information indicating that the second electronic device establishes a Bluetooth connection with the to-be-fitted hearing aid, displays, on an interactive interface, that an object has been connected, and displays acquired basic information of the to-be-fitted hearing aid, for example, a current remaining electric quantity of the hearing aid.

After the first electronic device controls the second electronic device to establish the Bluetooth connection with the hearing aid, the first electronic device updates, according to a change of Bluetooth signal strength information (for example, a signal strength), a fitting parameter for fitting the to-be-fitted hearing aid, and performs audiometry on the user by updating the fitting parameter. After receiving an audiometry result (a corresponding fitting result) of the user, the first electronic device calculates a configuration parameter of a corresponding hearing aid according to the audiometry result and Bluetooth connection signal strength information between the second electronic device and the to-be-fitted hearing aid. The first electronic device transmits the calculated configuration parameter of the hearing aid to the second electronic device, and the second electronic device configures the to-be-fitted hearing aid according to the configuration parameter of the hearing aid.

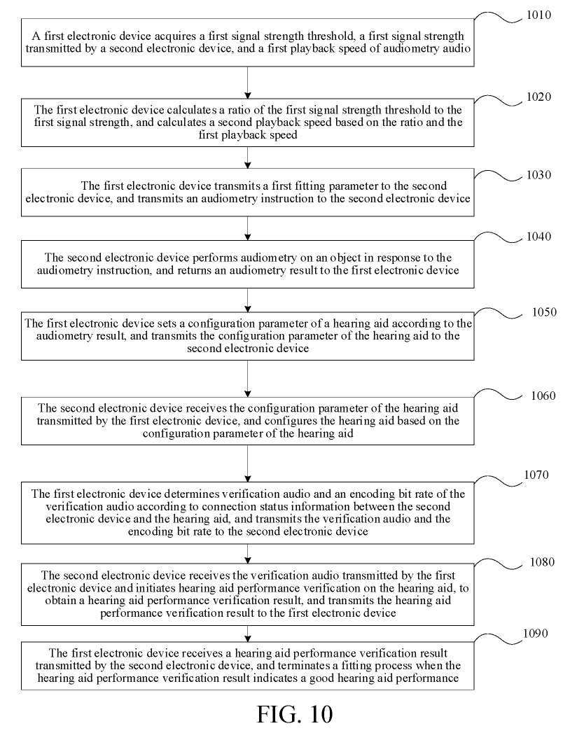

FIG. 10 is another schematic flowchart of a hearing aid fitting method according to an embodiment of this disclosure. The hearing aid fitting method includes the following operations:

Operation 1010: A first electronic device acquires a first signal strength threshold, a first signal strength transmitted by a second electronic device, and a first playback speed of audiometry audio.

In some embodiments, after the Bluetooth connection succeeds, the first electronic device acquires the first signal strength threshold, the first playback speed of the audiometry audio, and acquires the first signal strength from the second electronic device. For example, the first signal strength threshold is set to -50 dBm, the first playback speed is 1× speed, and the first signal strength acquired from the second electronic device is -75 dBm.

In some embodiments, an object client may further transmit a battery level of the hearing aid to the first electronic device. When the battery level of the hearing aid is lower than a preset reference battery level, the first electronic device may initiate, to the object client, a notification for charging the hearing aid, and detect the battery level of the hearing aid in real time, to improve integrity of a fitting process.

The first electronic device may alternatively adjust the first signal strength threshold and the first playback speed according to an environment in which the hearing aid is located. Different first signal strength thresholds and different first playback speeds may be set for different environments in which the hearing aid is located.

For operation 1010, refer to descriptions of performing environment detection based on the connection status information, to obtain a detection result, and determining, based on the connection status information in a case that the detection result indicates that an environmental condition satisfies a fitting condition, a first fitting parameter for fitting the hearing aid. Details are not described herein.

Operation 1020: The first electronic device calculates a ratio of the first signal strength threshold to the first signal strength, and calculates a second playback speed based on the ratio and the first playback speed.

In some embodiments, the first signal strength threshold is equal to the second signal strength threshold, the ratio of the first signal strength threshold to the first signal strength is calculated, and the second playback speed of audiometry audio is calculated according to the ratio and the first playback speed. The second playback speed of the audiometry audio may be a first fitting parameter for fitting the hearing aid.

In addition, the first electronic device may further adjust an encoding bit rate of the audiometry audio according to the first signal strength received from the second electronic device. In a case that the first signal strength is less than or equal to the first signal strength threshold, the first electronic device may reduce the encoding bit rate of the audiometry audio, to improve the transmission of the audiometry audio to the second electronic device. In a case that the first signal strength is greater than the first signal strength threshold, the first electronic device may properly increase the encoding bit rate of the audiometry audio, whereby the second electronic device may receive clearer and higher-quality audiometry audio, and user fitting experience is improved.

Operation 1030: The first electronic device transmits a first fitting parameter to the second electronic device, and transmits an audiometry instruction to the second electronic device.

In some embodiments, the first electronic device transmits the second playback speed and the encoding bit rate to the second electronic device, and transmits the audiometry instruction to the second electronic device. The audiometry instruction is configured to inform the second electronic device to control the hearing aid to perform audiometry on a user based on the second playback speed and the encoding bit rate (for example, the first fitting parameter).

After receiving the audiometry instruction, the second electronic device initiates audiometry to the hearing aid, and transmits the audiometry audio, the encoding bit rate, and the corresponding playback speed to the hearing aid, whereby the hearing aid performs, according to the audiometry audio, the encoding bit rate, and the playback speed, audiometry on the user with hearing impairment. The first electronic device may store, to the cloud, a fitting parameter debugged in a fitting process, and update and optimize a hearing aid fitting system according to a historical record of adjustment of the fitting parameter of the hearing aid, to improve a response capability and working efficiency of the hearing aid fitting system.

In some embodiments, in the process in which the second electronic device initiates the audiometry of the object to the hearing aid based on the fitting parameter, operation 1030 further includes:

receiving, by the first electronic device at preset duration, a second signal strength that is of the Bluetooth connection between the second electronic device and the hearing aid and that is returned by the second electronic device;

generating, by the first electronic device, a third fitting parameter when a difference between the second signal strength and the first signal strength is greater than a preset threshold; and

transmitting, by the first electronic device, the third fitting parameter to the second electronic device, and performing, by the second electronic device, audiometry on an object based on the third fitting parameter transmitted by the first electronic device.

The first electronic device may dynamically adjust the fitting parameter according to a Bluetooth connection signal strength between the second electronic device and the hearing aid, and reduce problems of inaccurate audiometry results caused by an unstable connection status, to improve accuracy of a hearing aid fitting system.

Operation 1040: The second electronic device performs audiometry on an object in response to the audiometry instruction, and returns an audiometry result to the first electronic device.

In some embodiments, after receiving the audiometry instruction transmitted by the first electronic device, the second electronic device initiates the audiometry to the hearing aid according to the fitting parameter, and the hearing aid performs the audiometry on the object client and generates the audiometry result. After the audiometry is completed, the second electronic device transmits a collected audiometry result to the first electronic device, and a fitting user evaluates and analyzes the audiometry result.

In some embodiments, alternatively, the first electronic device may automatically generate a configuration parameter setting template of the hearing aid according to the audiometry result and the first signal strength, and automatically fill in the configuration parameter of the hearing aid that is recommended according to the audiometry result and the first signal strength. The configuration parameter setting template of the hearing aid is displayed on a fitting application software interface of the first electronic device. Then, the fitter user evaluates reasonableness of the configuration parameter of the hearing aid automatically generated by the first electronic device, to determine the configuration parameter of the hearing aid that needs to be finally transmitted to the second electronic device.

Operation 1050: The first electronic device sets a configuration parameter of a hearing aid according to the audiometry result, and transmits the configuration parameter of the hearing aid to the second electronic device.

In some embodiments, the first electronic device receives the audiometry result transmitted by the second electronic device, and after analyzing the audiometry result, the fitting user sets the configuration parameter of the hearing aid by using the first electronic device. Alternatively, the first electronic device may analyze the audiometry result and generate a corresponding configuration parameter of the hearing aid, and then the fitting user evaluates whether the configuration parameter of the hearing aid generated by fitting software is proper. If an improper parameter setting exists, manual adjustment is performed. For example, if the compensation gain of the hearing aid is excessively high, the hearing loss of the object user may be worsened. In this case, the compensation gain of the hearing aid needs to be properly reduced.

The configuration parameter of the hearing aid may include parameters such as a sound gain, volume setting, audio noise reduction, and feedback suppression of the hearing aid. By adjusting the configuration parameter of the hearing aid, hearing of the user with hearing impairment may be compensated. There may be a plurality of manners of setting the configuration parameter of the hearing aid. For example, the configuration parameter may be manually input into the first electronic device by the fitter user, or may be selected by the fitter user by selecting a corresponding fitting parameter setting template in software of the first electronic device. Both the first electronic device and the second electronic device support a voice operation. The first electronic device may input a fitting parameter and the configuration parameter of the hearing aid by using a voice. After receiving the fitting parameter and the configuration parameter of the hearing aid that are transmitted by the first electronic device, the second electronic device may alternatively play the fitting parameter and the configuration parameter of the hearing aid to the user in a voice broadcasting form. In some embodiments, after generating the configuration parameter of the hearing aid, the first electronic device encapsulates the configuration parameter of the hearing aid and transmits the encapsulated configuration parameter to the second electronic device by using an instant message channel.

Operation 1060: The second electronic device receives the configuration parameter of the hearing aid transmitted by the first electronic device, and configures the hearing aid based on the configuration parameter of the hearing aid.

In some embodiments, the second electronic device receives and decapsulates the configuration parameter of the hearing aid transmitted by the first electronic device, and transmits the configuration parameter of the hearing aid to the hearing aid by using a Bluetooth wireless connection channel, to configure the hearing aid. The hearing aid operates according to an input performance parameter, to compensate for the hearing loss of the user.

Operation 1070: The first electronic device determines verification audio and an encoding bit rate of the verification audio according to connection status information between the second electronic device and the hearing aid, and transmits the verification audio and the encoding bit rate to the second electronic device.

In some embodiments, after the hearing aid operates for a particular time according to a set parameter, and when the user can adapt to a current operating mode of the hearing aid, the first electronic device detects a hearing performance of the hearing aid, and the second electronic device alternatively transmits a current first signal strength between the second electronic device and the hearing aid to the first electronic device in real time. The first electronic device receives the first signal strength transmitted by the second electronic device, and generates the verification audio. The playback speed of the verification audio is generated after calculating and updating according to the Bluetooth connection signal strength.

When the Bluetooth connection signal strength (for example, the first signal strength) is relatively low (the first signal strength is less than or equal to the first signal strength threshold), the encoding bit rate of the verification audio may be reduced, to enhance transmission stability, and the object client may receive the verification audio. When the Bluetooth connection signal strength is relatively high (the first signal strength is greater than the first signal strength threshold), the encoding bit rate of the verification audio may be improved, and the user may obtain verification audio of higher quality. When the Bluetooth connection signal strength maintains at a relatively stable value, the first electronic device does not need to adjust the encoding bit rate of the verification audio. The first electronic device transmits the verification audio, the set playback speed information, and the encoding bit rate to the second electronic device. In some embodiments, the first electronic device may further calculate the playback speed of the verification audio according to the method for calculating the playback speed of the audiometry audio in the foregoing operation, whereby the playback speed of the verification audio may better adapt to a current Bluetooth connection signal strength. Therefore, whether a current hearing aid speed of the hearing aid meets an actual requirement of the user may be accurately verified, to obtain an accurate hearing understanding capability of the user.

Operation 1080: The second electronic device receives the verification audio transmitted by the first electronic device and initiates hearing aid performance verification on the hearing aid, to obtain a hearing aid performance verification result, and transmits the hearing aid performance verification result to the first electronic device.

In some embodiments, after receiving the verification audio and corresponding playback speed information that are transmitted by the first electronic device, the second electronic device initiates a hearing aid performance verification instruction for the hearing aid. The hearing aid performance verification instruction may include the verification audio, the playback speed, and the encoding bit rate. After receiving the hearing aid performance verification instruction transmitted by the second electronic device, the hearing aid performs hearing aid performance verification on the object user according to the verification audio. In this process, the first electronic device may be in contact with the user by using a real-time audio communication technology such as video software, whereby the fitting user acquires a feedback result of the user by observing facial expressions and body responses of the user by using the video software.

If the user has any discomfort during the hearing aid performance verification process, the fitting user may be informed instantly by using the video software, and the first electronic device terminates the hearing aid performance verification process. After waiting for the verification audio to be played, the second electronic device receives a verification result of the hearing aid performance, and transmits the verification result of the hearing aid performance to the first electronic device.

Operation 1090: The first electronic device receives a hearing aid performance verification result transmitted by the second electronic device, and terminates a fitting process when the hearing aid performance verification result indicates a good hearing aid performance.

In some embodiments, the first electronic device receives the hearing aid performance verification result (for example, the verification result) returned by the second electronic device. The fitting user analyzes the hearing aid performance verification result, acquires real-time feedback of the user by communicating with the user and by using the video, and determines whether the current hearing aid performance is good. Alternatively, the hearing aid performance verification result may be analyzed by using software of the first electronic device.

In some embodiments, the first electronic device may evaluate and analyze verification results, such as whether the hearing aid solves a hearing problem of the user with hearing impairment reflected in a hearing report, whether the user with hearing impairment is able to hear clear sound, whether content of the sound and information conveyed by the sound is able to be correctly understood after the sound is heard, whether a noise reduction function in a noisy environment is good, and whether a hearing experience in a listening process is comfortable, under a current configuration parameter of the hearing aid. When the hearing aid performance verification result indicates a good hearing aid performance, a message pop-up window is generated on an interactive interface (for example, a display screen of the first electronic device), to notify the fitting user that the hearing aid performance verification result indicates a good hearing aid performance. The first electronic device may terminate the fitting process and transmit, to the second electronic device, a message notification indicating that the fitting process is terminated. After receiving the message notification indicating that the fitting is terminated, the second electronic device automatically disconnects the Bluetooth connection from the hearing aid. When the hearing aid performance verification result indicates a poor hearing aid performance, the software of the first electronic device notifies the fitting user that the hearing aid performance verification result indicates that the hearing aid performance is poor, fitting needs to be performed again, a message instruction for re-performing fitting on the hearing aid is transmitted to the second electronic device, and the foregoing hearing aid fitting operations are repeated.

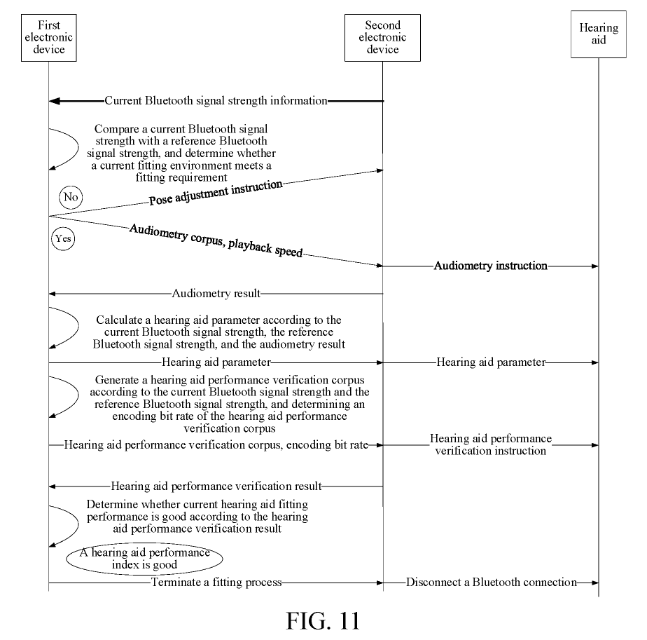

FIG. 11 is a schematic diagram of data transmission in a fitting process of a hearing aid according to an embodiment of this disclosure. In some aspects, data transmission performed by three ends, namely, a first electronic device, a second electronic device, and a hearing aid, after a fitting process is started after establishing a Bluetooth connection between the second electronic device and the hearing aid. The transmission process is as follows:

In some embodiments, after the Bluetooth connection between the hearing aid and the second electronic device is established, the first electronic device receives, by using the instant message channel at regular intervals, the Bluetooth signal strength information (for example, the first signal strength) transmitted by the second electronic device. After receiving the Bluetooth signal strength information, the first electronic device compares a current Bluetooth signal strength with a preset reference Bluetooth signal strength (for example, the first signal strength threshold). If a Bluetooth signal strength value is not greater than a preset Bluetooth signal strength value in this case, a current fitting environment does not meet a fitting requirement. The first electronic device transmits a pose adjustment instruction to the second electronic device. The pose adjustment instruction may be a text instruction, a voice instruction, or a graphic instruction, to notify the user to move the hearing aid close to the second electronic device, or move away from a physical obstacle and an electromagnetic interference device, to perform testing and fitting in a relatively quiet environment. The user performs corresponding pose adjustment according to the notification transmitted by the second electronic device, until the Bluetooth connection signal strength received by the first electronic device satisfies the reference Bluetooth signal strength value, and the first electronic device transmits, to the second electronic device, an instruction indicating that a current fitting environment has already met a fitting requirement, and audiometry will be started.

In some embodiments, if the Bluetooth signal strength value is greater than the reference Bluetooth signal strength value in this case, the current fitting environment meets a fitting requirement, and a next fitting operation may be performed (for example, the audiometry is performed on the user). The first electronic device receives the Bluetooth signal strength information transmitted by the second electronic device, and calculates a playback speed of the audiometry audio according to a ratio of the current Bluetooth signal strength to the reference Bluetooth signal strength. The method for calculating the playback speed has been described in the foregoing operations, and details are not described herein again.

In some embodiments, the first electronic device may further adjust an encoding bit rate of the audiometry audio according to current Bluetooth connection signal strength information. When the Bluetooth connection signal strength (for example, the second signal strength) received by the first electronic device is lower than the Bluetooth connection signal strength (for example, the first signal strength) received previously, the first electronic device needs to correspondingly reduce the encoding bit rate of the audiometry audio. When the Bluetooth connection signal strength received by the first electronic device is higher than the Bluetooth connection signal strength received previously, the first electronic device needs to correspondingly increase the encoding bit rate of the audiometry audio. If the current Bluetooth connection signal strength remains consistent with the Bluetooth connection signal strength received previously, the current encoding bit rate of the audiometry audio is maintained.

The first electronic device encapsulates the audiometry audio, the corresponding playback speed, and the encoding bit rate into an instant message and wirelessly transmits the instant message to the second electronic device. The second electronic device initiates audiometry to the hearing aid according to the received audiometry audio and playback speed, and transmits the audiometry audio, the playback speed, and the encoding bit rate to the hearing aid. In the foregoing process, the first electronic device receives the Bluetooth connection signal strength information (for example, the second signal strength) between the second electronic device and the hearing aid at regular intervals, and calculates and updates the fitting parameter according to the Bluetooth connection signal strength information, the reference playback speed, and a reference encoding bit rate, to obtain a target playback speed and a target encoding bit rate of the audiometry audio at the current Bluetooth connection signal strength, to improve accuracy of an audiometry result. The first electronic device transmits the updated fitting parameter to the second electronic device, to control the second electronic device to perform audiometry on the user.

Then, the second electronic device transmits the audiometry result to the first electronic device by using the instant message channel. After receiving the audiometry result, the first electronic device analyzes the audiometry result to obtain analysis results such as a degree of hearing loss of the user and sound frequencies at which hearing performance is relatively poor. The first electronic device calculates the configuration parameter of the hearing aid according to the analysis results and the current Bluetooth signal strength.

In some embodiments, the first electronic device transmits the calculated configuration parameter of the hearing aid to the second electronic device by using the instant message channel, and the second electronic device receives the configuration parameter of the hearing aid transmitted by the first electronic device and configures the hearing aid according to the configuration parameter of the hearing aid. The second electronic device transmits the configuration parameter of the hearing aid to the hearing aid by using the Bluetooth wireless connection channel of the hearing aid. After transmission of the configuration parameter of the hearing aid is completed, the second electronic device transmits, to the first electronic device, an instruction that indicates that the configuration parameter of the hearing aid has been configured and the current Bluetooth connection signal strength information.

After receiving the instruction that indicates that the configuration parameter of the hearing aid has been configured and that is transmitted by the second electronic device, the first electronic device performs a next fitting operation (for example, performs verification on a hearing aid performance of the current hearing aid). The first electronic device generates a verification audio according to the Bluetooth connection signal strength information transmitted back by the second electronic device, and calculates an encoding bit rate of the verification audio according to the current Bluetooth signal strength information. In addition, the first electronic device may alternatively calculate the playback speed of the verification audio according to a ratio of the current Bluetooth connection signal strength to the reference Bluetooth signal strength. A method for calculating the playback speed of the verification audio is the same as a method for calculating the playback speed of the audiometry audio, and details are not described herein again. The first electronic device adjusts the playback speed and the encoding bit rate of the verification audio by acquiring the Bluetooth connection signal strength information transmitted by the second electronic device, to acquire a more accurate hearing aid performance verification result, to improve experience of the user in a fitting process.

In some embodiments, the first electronic device may transmit the calculated verification audio and encoding bit rate, and the corresponding playback speed to the second electronic device by using an instant message channel. After receiving the verification audio, the encoding bit rate, and the playback speed, the second electronic device initiates a hearing aid performance verification instruction to the hearing aid. The hearing aid performance verification instruction includes the verification audio, the encoding bit rate, and the playback speed. After the second electronic device completes hearing aid performance verification on the hearing aid, the second electronic device transmits a hearing aid performance verification result to the first electronic device. The first electronic device receives and parses the hearing aid performance verification result, and analyzes whether the hearing aid performance of the current hearing aid reaches a good index. If a current hearing aid performance index is good, the first electronic device transmits an instruction for terminating a fitting process to the second electronic device. In addition, the fitting information, including a fitting parameter of the hearing aid, user feedback, and an audiometry result of the user, in this fitting process is uploaded to a cloud for storage, whereby the first electronic device may acquire historical fitting information from the cloud to develop a fitting solution during next fitting. After receiving an instruction for terminating the fitting process, the second electronic device notifies that the current fitting performance is good on the second electronic device, and terminates the fitting process. In addition, the second electronic device actively disconnects a Bluetooth connection from the hearing aid, to complete remote fitting of the hearing aid.

Descriptions of an apparatus and a device in this disclosure

Although the operations are displayed sequentially according to the indications of the arrows in the flowcharts of the embodiments, these operations are not necessarily performed sequentially in the sequence indicated by the arrows. Unless otherwise explicitly specified in the embodiments, execution of the operations is not strictly limited, and the operations may be performed in other sequences. Moreover, at least some of the operations in the flowcharts may include a plurality of operations or a plurality of stages. The operations or stages are not necessarily performed at the same time but may be performed at different time. Execution of the operations or stages is not necessarily sequentially performed, but may be performed in turn or alternately with other operations or at least some of operations or stages of other operations.

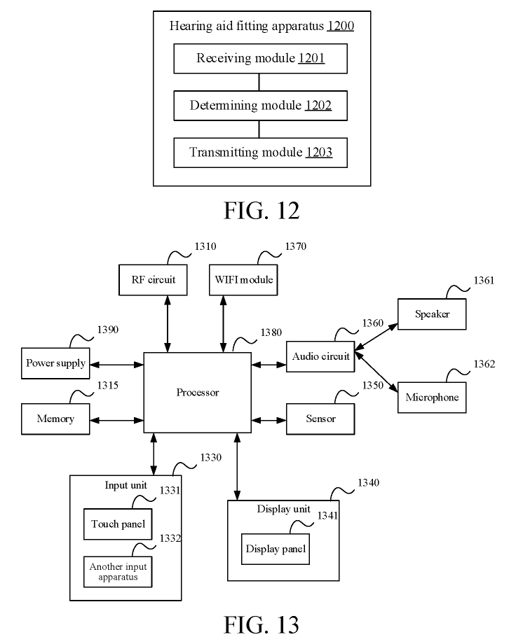

FIG. 12 is a schematic structural diagram of a hearing aid fitting apparatus according to an embodiment of this disclosure. FIG. 12 shows a hearing aid fitting apparatus 1200. The hearing aid fitting apparatus 1200 includes:

a receiving module 1201, configured to receive, by a first electronic device, connection status information transmitted by a second electronic device, the connection status information being configured to indicate a connection status between the second electronic device and a hearing aid, and the second electronic device being communicatively coupled to the hearing aid based on short-range wireless communication;

a determining module 1202, configured to determine, based on the connection status information, a first fitting parameter for fitting the hearing aid; and

a transmitting module 1203, configured to transmit the first fitting parameter to the second electronic device, to trigger the second electronic device to fit, based on the first fitting parameter, the hearing aid.

In some embodiments, the determining module 1202 is configured to: perform environment detection based on the connection status information, to obtain a detection result; and determine, based on the connection status information in a case that the detection result indicates that an environmental condition satisfies a fitting condition, the first fitting parameter for fitting the hearing aid.

In some embodiments, the connection status information includes a first signal strength; and the determining module 1202 is configured to: acquire a first signal strength threshold of the hearing aid; perform environment detection based on the first signal strength and the first signal strength threshold; and determine, in a case that the first signal strength is greater than the first signal strength threshold, that the detection result is that the environmental condition satisfies the fitting condition.

In some embodiments, the transmitting module 1203 is configured to: generate guidance information based on the connection status information in a case that the detection result indicates that the environmental condition does not satisfy the fitting condition, the guidance information being configured to indicate an adjustment to a relative position between the second electronic device and the hearing aid; and transmit the guidance information to the second electronic device, to trigger the second electronic device to display the guidance information.

In some embodiments, the connection status information includes a first signal strength; and the determining module 1202 is configured to: acquire a second signal strength threshold and a second fitting parameter of the hearing aid; determine a ratio of the first signal strength to the second signal strength threshold; and determine, based on the ratio and the second fitting parameter, the first fitting parameter for fitting the hearing aid.

In some embodiments, the second fitting parameter includes a first playback speed of audiometry audio; and the determining module 1202 is configured to: determine a second playback speed of the audiometry audio based on the first playback speed and the ratio after transmitting the first fitting parameter to the second electronic device; and determine, based on the second playback speed, the first fitting parameter for fitting the hearing aid.

In some embodiments, the determining module 1202 is configured to: determine verification audio based on the connection status information after transmitting a configuration parameter to the second electronic device, and determine an encoding parameter for the verification audio, the verification audio being configured to verify performance of the hearing aid after being configured by using the configuration parameter; generate a verification request based on the verification audio and the encoding parameter; and transmit the verification request to the second electronic device, to trigger the second electronic device to verify the performance of the configured hearing aid based on the verification audio in the verification request.

In some embodiments, the transmitting module 1203 is configured to: periodically receive, after transmitting the first fitting parameter to the second electronic device, a second signal strength transmitted by the second electronic device, the second signal strength being transmitted by the second electronic device in a process of fitting, based on the first fitting parameter, the hearing aid; determine a difference between the second signal strength and the first signal strength; generate a third fitting parameter when the difference is greater than a preset difference threshold; and transmit the third fitting parameter to the second electronic device, to trigger the second electronic device to replace the first fitting parameter with the third fitting parameter.

In some embodiments, the connection status information includes a third signal strength of the short-range wireless communication at a first moment and a fourth signal strength of the short-range wireless communication at a second moment, and the first moment is earlier than the second moment; and the determining module 1202 is configured to: acquire a difference between the third signal strength and the fourth signal strength; determine, based on the fourth signal strength in a case that the difference is greater than the difference threshold, the first fitting parameter for fitting the hearing aid; and determine, based on the third signal strength in a case that the difference is less than or equal to the difference threshold, the first fitting parameter for fitting the hearing aid.

In some embodiments, the transmitting module 1203 is configured to: transmit, by the first electronic device before the first electronic device receives the connection status information transmitted by the second electronic device, a fitting request to the second electronic device, the fitting request being configured to trigger the second electronic device to initiate a device search process in compliant with a short-range wireless communication protocol (e.g., a Bluetooth search); receive a plurality of pieces of search information transmitted by the second electronic device; generate a connection request based on a target piece of search information corresponding to the hearing aid when the plurality of pieces of search information include the target piece of search information corresponding to the hearing aid; and transmit the connection request to the second electronic device, to trigger the second electronic device to establish a short-range wireless connection compliant with the short-range wireless communication protocol (e.g., a Bluetooth connection) with the hearing aid based on the target piece of search information corresponding to the hearing aid in the connection request.

In some embodiments, the transmitting module 1203 is configured to: display the plurality of pieces of search information, the plurality of pieces of search information including the target piece of search information corresponding to the hearing aid; and generate the connection request based on the target piece of search information corresponding to the hearing aid in response to a selection operation for the target piece of search information corresponding to the hearing aid.

In some embodiments, the transmitting module 1203 is configured to: receive, before receiving the plurality of pieces of search information transmitted by the second electronic device, information of the hearing aid transmitted by the second electronic device; and transmit a start request to the hearing aid based on the information of the hearing aid, to trigger the hearing aid to activate a communication functionality compliant with the short-range wireless communication protocol (e.g., Bluetooth functionality) based on the start request.

In this disclosure, the term "module" or "unit" refers to a computer program with a preset function or a part of the computer program and works, together with other related parts, to implement a preset target, and may be implemented by using software, hardware (for example, processing circuitry or a memory) or a combination thereof. Similarly, one processor (or a plurality of processors or memories) may be configured to implement one or more modules or units. In addition, each module or unit may be a part of an overall module or unit including a function of the module or unit.

FIG. 13 is a schematic structural diagram of an electronic device according to an embodiment of this disclosure. FIG. 13 may be a schematic diagram of a part of a structure of a first electronic device 110 or a second electronic device 120 of a hearing aid fitting method according to an embodiment of this disclosure. The first electronic device 110 or the second electronic device 120 includes components such as a radio frequency (RF) circuit 1310, a memory 1315 (for example, including a non-transitory computer-readable storage medium), an input unit 1330, a display unit 1340, a sensor 1350, an audio circuit 1360, a Wi-Fi module 1370, processing circuitry (for example, a processor 1380), and a power supply 1390. A person skilled in the art may understand that a structure of the first electronic device 110 or the second electronic device 120 shown in FIG. 13 does not constitute a limitation to a mobile phone or a computer, and may include more components or fewer components than those shown in the figure, or some components may be combined, or a different component deployment may be used.

The RF circuit 1310 may be configured to receive and transmit signals during an information receiving and transmitting process or a call process, and particularly, to receive downlink information from a base station for the processor 1380 to process. In addition, an uplink data transmitting base station is designed.

The memory 1315 may include a non-transitory computer-readable storage medium configured to store a software program and a module, and the processor 1380 executes various function applications of the terminal and performs document editing by running the software program and the module stored in the memory 1315.

The input unit 1330 may be configured to receive input numerical or character information, and generate a key signal input related to terminal setting and function control. In some embodiments, the input unit 1330 may include a touch panel 1331 and another input apparatus 1332.

The display unit 1340 may be configured to display input information or provided information and various menus of the terminal. The display unit 1340 may include a display panel 1341.

The audio circuit 1360, a speaker 1361, and a microphone 1362 may provide audio interfaces.

In this embodiment, the processor 1380 included in the first electronic device 110 and the second electronic device 120 may perform the hearing aid fitting method according to one or more embodiments described in this disclosure.

The first electronic device 110 and the second electronic device 120 in this disclosure include, but are not limited to, a mobile phone, a computer, a smart voice interaction device, a smart home appliance, an in-vehicle terminal, an aircraft, and the like.

Embodiments of this disclosure further provide a non-transitory computer-readable storage medium, the non-transitory computer-readable storage medium being configured to store program code, and the program code being configured to perform the hearing aid fitting method according to the foregoing embodiments.

An embodiment of this disclosure further provides a computer program product, and the computer program product includes a computer program. A processor of an electronic device reads and executes the computer program, to enable the electronic device to implement the foregoing hearing aid fitting method.

Terms such as "first", "second", "third", and "fourth" (if exist) in the specification of this disclosure and in the accompanying drawings are used for distinguishing between similar objects and not necessarily used for describing any particular order or sequence. Data used in this way is exchangeable in a proper case, so that the embodiments of this disclosure described herein may be implemented in a sequence different from a sequence shown or described herein. In addition, terms "comprise" and "include" and any other variants are intended to cover a non-exclusive inclusion. For example, a process, method, system, product, or apparatus that includes a series of operations or units is not necessarily limited to those operations or units that are expressly listed, but may include other operations or units that are not expressly listed or inherent to such a process, method, product, or apparatus.

In this embodiment of this disclosure, "at least one (item)" refers to one or more, and "a plurality of" refers to two or more. Term "and/or" is configured to describe an association relationship between associated objects and indicates that three relationships may exist. For example, "A and/or B" may indicate the following three cases: only A exists, only B exists, and both A and B exist. A and B may be singular or plural. The character "/" indicates an "or" relationship between the associated objects. "At least one item (piece) of the following" or a similar expression refers to any combination of these items, including a single item (piece) or any combination of a plurality of items (pieces). For example, at least one of a, b, or c may represent: a, b, c, "a and b", "a and c", "b and c", or "a and b and c", where a, b, and c may be singular or plural.

In the descriptions of one or more embodiments of this disclosure, a plurality of pieces (or a plurality of items) refers to two or more, greater than, less than, and exceeding, and the like are understood as excluding the number, and above, below, within, and the like are understood as including the number.

In one or more embodiments provided in this disclosure, the disclosed system, apparatus, and method may be implemented in other manners. For example, the apparatus embodiment described above is schematic, for example, division of units is logic function division, and other division manners may be adopted during practical implementation. For example, a plurality of units or components may be combined or integrated into another system, or some features may be neglected or not executed. In addition, the displayed or discussed mutual couplings or direct couplings or communication connections may be implemented by using some interfaces. The indirect couplings or communication connections between the apparatuses or units may be implemented in electronic, mechanical, or other forms.

Units described as separate components may or may not be physically separate, and components displayed as units may or may not be physical units (for example, may be located in one place or distributed across a plurality of network units). Part or all of the units may be selected according to actual needs to achieve purposes of solutions of the embodiments.

In addition, functional units in one or more embodiments of this disclosure may be integrated into one processing unit, or each unit may exist alone physically, or two or more units may be integrated into one unit. The foregoing integrated unit may be implemented in a form of hardware, or may be implemented in a form of a software functional unit.

When implemented in a form of a software functional unit and sold or used as an independent product, the integrated unit may alternatively be stored in a storage medium. Based on such an understanding, technical solutions of this disclosure may be implemented in a form of a software product. The computer software product is stored in a non-transitory computer-readable storage medium and includes a plurality of instructions for indicating a computer apparatus (which may be a personal computer, a server, a network apparatus, or the like) to perform all or part operations of the methods described in one or more embodiments of this disclosure. The foregoing storage medium includes: media that can store program code, such as a universal serial bus (USB) flash drive, a mobile hard disc drive, a read-only memory (ROM), a random access memory (RAM), a magnetic disc, or an optical disc.

Various implementations provided in one or more embodiments of this disclosure may be combined in different manners to form other embodiments.

The foregoing describes non-limiting examples of the implementations of this disclosure, but this disclosure is not limited to the foregoing implementations. A person skilled in the art may further make various modifications or replacements without departing from the spirit of this disclosure, and these modifications or replacements are included within the scope of this disclosure.

What is claimed is:

1. A hearing aid fitting method, comprising:
receiving, by processing circuitry of a first electronic device, connection status information indicating a connection status between a second electronic device and a hearing aid, the second electronic device being communicatively coupled to the hearing aid via wireless communication;
determining a detection result indicating whether an environmental condition based on the connection status information satisfies a fitting condition;
determining, by the processing circuitry based on the connection status information, a first fitting parameter for fitting the hearing aid when the detection result indicates that the environmental condition satisfies the fitting condition; and
transmitting the first fitting parameter to the second electronic device that is configured to engage in a fitting process with the hearing aid based on the first fitting parameter.

2. The method according to claim 1, wherein
the connection status information includes a first signal strength, and
the determining the detection result includes:
setting the detection result to indicate that the environmental condition satisfies the fitting condition when the first signal strength is greater than a first signal strength threshold associated with the hearing aid.

3. The method according to claim 1, further comprising:
generating guidance information based on the connection status information when the detection result indicates that the environmental condition does not satisfy the fitting condition, the guidance information indicating an adjustment to a relative position between the second electronic device and the hearing aid; and
transmitting the guidance information to the second electronic device that is configured to display the guidance information.

4. The method according to claim 1, wherein
the connection status information includes a first signal strength, and
the determining the first fitting parameter includes:
determining a ratio of the first signal strength to a second signal strength threshold associated with the hearing aid; and
determining the first fitting parameter for fitting the hearing aid based on a second fitting parameter and the ratio.

5. The method according to claim 4, wherein
the second fitting parameter includes a first playback speed of audiometry audio, and
the determining the first fitting parameter for fitting the hearing aid based on the second fitting parameter and the ratio includes:
determining a second playback speed of the audiometry audio based on the first playback speed and the ratio; and
determining the first fitting parameter for fitting the hearing aid based on the second playback speed.

6. The method according to claim 1, further comprising:
receiving a fitting result of the fitting process;
generating a configuration parameter of the hearing aid based on the fitting result; and
transmitting the configuration parameter to the second electronic device that is configured to configure the hearing aid based on the configuration parameter.

7. The method according to claim 6, further comprising:
determining verification audio based on the connection status information;
determining an encoding parameter for the verification audio, the verification audio being usable to verify performance of the hearing aid configured based on the configuration parameter;
generating a verification request based on the verification audio and the encoding parameter; and
transmitting the verification request to the second electronic device that is configured to verify the performance of the hearing aid based on the verification audio in the verification request.

8. The method according to claim 1, further comprising:
receiving a second signal strength indicating the connection status during the fitting process;
determining a difference between the second signal strength and a first signal strength included in the connection status information;
generating a third fitting parameter when the difference is greater than a preset difference threshold; and
transmitting the third fitting parameter to the second electronic device that is configured to replace the first fitting parameter with the third fitting parameter.

9. The method according to claim 1, wherein
the connection status information includes a third signal strength of the wireless communication at a first moment and a fourth signal strength of the wireless communication at a second moment, the first moment being earlier than the second moment; and
the determining the first fitting parameter includes:
obtaining a difference between the third signal strength and the fourth signal strength;
determining the first fitting parameter based on the fourth signal strength when the difference is greater than a difference threshold; and
determining the first fitting parameter, based on the third signal strength when the difference is less than or equal to the difference threshold.

10. The method according to claim 1, further comprising:
transmitting a fitting request to the second electronic device that is configured to initiate a device search process compliant with a short-range wireless communication protocol based on the fitting request;
receiving a plurality of pieces of search information corresponding to the device search process;
generating a connection request based on a target piece of search information corresponding to the hearing aid from the plurality of pieces of search information; and
transmitting the connection request to the second electronic device that is configured to establish a short-range wireless connection compliant with a short-range wireless communication protocol with the hearing aid based on the target piece of search information corresponding to the hearing aid in the connection request.

11. The method according to claim 10, wherein the generating the connection request comprises:
displaying the plurality of pieces of search information, the plurality of pieces of search information including the target piece of search information corresponding to the hearing aid; and
generating the connection request based on a selection operation of the target piece of search information corresponding to the hearing aid.

12. The method according to claim 10, further comprising:
receiving information of the hearing aid; and
transmitting a start request to the hearing aid based on the information of the hearing aid that is configured to activate a communication functionality compliant with the short-range wireless communication protocol based on the start request.

13. A hearing aid fitting apparatus, comprising:
processing circuitry configured to:
receive connection status information indicating a connection status between an electronic device and a hearing aid, the electronic device being communicatively coupled to the hearing aid via wireless communication;
determine a detection result indicating whether an environmental condition based on the connection status information satisfies a fitting condition;
determine, based on the connection status information, a first fitting parameter for fitting the hearing aid when the detection result indicates that the environmental condition satisfies the fitting condition; and
transmit the first fitting parameter to the electronic device that is configured to engage in a fitting process with the hearing aid based on the first fitting parameter.

14. The apparatus according to claim 13, wherein
the connection status information includes a first signal strength, and
the processing circuitry is configured to:
set the detection result to indicate that the environmental condition satisfies the fitting condition when the first signal strength is greater than a first signal strength threshold associated with the hearing aid.

15. The apparatus according to claim 13, wherein the processing circuitry is configured to:
generate guidance information based on the connection status information when the detection result indicates that the environmental condition does not satisfy the fitting condition, the guidance information indicating an adjustment to a relative position between the electronic device and the hearing aid; and
transmit the guidance information to the electronic device that is configured to display the guidance information.

16. The apparatus according to claim 13, wherein
the connection status information includes a first signal strength, and
the processing circuitry is configured to:
determine a ratio of the first signal strength to a second signal strength threshold associated with the hearing aid; and
determine the first fitting parameter for fitting the hearing aid based on a second fitting parameter and the ratio.

17. The apparatus according to claim 13, wherein the processing circuitry is configured to:
receive a fitting result of the fitting process;
generate a configuration parameter of the hearing aid based on the fitting result; and
transmit the configuration parameter to the electronic device that is configured to configure the hearing aid based on the configuration parameter.

18. A non-transitory computer-readable storage medium storing instructions, which when executed by a processor, cause the processor to perform a hearing aid fitting method, the method comprising:
receiving connection status information indicating a connection status between an electronic device and a hearing aid, the electronic device being communicatively coupled to the hearing aid via wireless communication;
determining a detection result indicating whether an environmental condition based on the connection status information satisfies a fitting condition;
determining, based on the connection status information, a first fitting parameter for fitting the hearing aid when the detection result indicates that the environmental condition satisfies the fitting condition; and
transmitting the first fitting parameter to the electronic device that is configured to engage in a fitting process with the hearing aid based on the first fitting parameter.

19. The non-transitory computer-readable storage medium according to claim 18, wherein
the connection status information includes a first signal strength, and
the determining the detection result includes:
setting the detection result to indicate that the environmental condition satisfies the fitting condition when the first signal strength is greater than a first signal strength threshold associated with the hearing aid.

20. The non-transitory computer-readable storage medium according to claim 18, wherein the method comprises:
generating guidance information based on the connection status information when the detection result indicates that the environmental condition does not satisfy the fitting condition, the guidance information indicating an adjustment to a relative position between the electronic device and the hearing aid; and
transmitting the guidance information to the electronic device that is configured to display the guidance information.
